(19) United States
(12) Patent Application Publication
(10) Pub. No.: US 2017/0292139 A1
(43) Pub. Date: Oct. 12, 2017

(54) GENETICALLY EXPANDED CELL FREE PROTEIN SYNTHESIS SYSTEMS, METHODS AND KITS

(71) Applicants: Lital ALFONTA, Omer (IL); Yonatan CHEMLA, Beersheba (IL); Eden OZER, Rishon Letzion (IL)

(72) Inventors: Lital ALFONTA, Omer (IL); Yonatan CHEMLA, Beersheba (IL); Eden OZER, Rishon Letzion (IL)

(73) Assignee: B.G NEGEV TECHNOLOGIES AND APPLICATIONS LTD., at BEN-GURION UNIVERSITY, Beersheba (IL)

(21) Appl. No.: 15/320,714

(22) PCT Filed: Jun. 17, 2015
(86) PCT No.: PCT/IL2015/050616

§ 371 Date: Dec. 20, 2016

Related U.S. Application Data
(60) Provisional application No. 62/180,143, filed on Jun. 16, 2015.
(60) Provisional application No. 62/013,024, filed on Jun. 17, 2014.

(51) Int. Cl. C12P 21/00; C12N 9/00

(52) U.S. Cl. CPC C12P 21/00; C12N 9/93; C12Y 601/01026; C12Y 601/01001

(57) ABSTRACT

This invention relates to methods of producing a rare amino acid- or non-natural amino acid-containing protein in a cell free protein synthesis system and kits for use in and for accomplishing same. Specifically, the methods comprise the steps of expressing at least one orthogonal suppressor tRNA (o-tRNA)/aminoacyl-tRNA synthetase (aaRS) pair or derivatives thereof specific for incorporation of a rare amino acid- or non-natural amino acid in an E. coli organism; preparing a lysate of said E. coli organism expressing said orthogonal suppressor tRNA (o-tRNA)/aminoacyl-tRNA synthetase (aaRS) pair; and contacting said lysate with a template DNA containing a mutant gene in which at least one amino acid codon at a given site of the protein-encoding gene has been mutated into an amber or ochre mutation and further providing a cognate rare amino acid or non-natural amino acid and other factors necessary for protein synthesis; wherein protein synthesis occurs following said contact to produce a protein containing said at least one rare amino acid or said non-natural amino acid. Kits for use are described, as well.

### FIELD OF THE INVENTION

This invention relates to methods of producing a rare amino acid- or non-natural amino acid-containing protein in a cell free protein synthesis system and kits for use in and for accomplishing same.

### BACKGROUND OF THE INVENTION

The ability to produce a mature and functional protein without the integrity of a living cell is the fundamental principal of cell free protein synthesis (CFPS). Of late, there is a marked increase in the development of cell free protein synthesis (CFPS) systems using systems that are diverse in function and with broad potential applications. This surge in CFP systems could be attributed to their unique advantages over in vivo methodologies.

Among the advantages to CFPs are: fast expression of recombinant proteins from DNA templates; High relative yields of up to 2.3 mg/ml of produced protein; Product parallel screening without the time consuming and gene-cloning step becomes possible; The absence of integral cells allows the manipulation of the micro-environment, the production and use of toxic and membrane impermeable molecules; and facile monitoring that enables immediate feedback and the manipulation of the protein synthesis process, to name a few. Due to the above mentioned advantages, the use of CFPS methodologies expanded, improved, made more accessible and are nowadays used for production of “hard-to-express” proteins, amino acid replacement, evolutionary biology, enzyme bioengineering, biotechnology and synthetic biology.

“Genetic code expansion” is the experimental attempt to increase the number of amino-acids repertoire that can be incorporated into proteins during ribosome-mediated translation. This increased repertoire enables the site-specific introduction of new chemical and physical properties into proteins—resulting in “enhanced proteins”—(i.e. proteins with one or more incorporated UAAs).

To date, there have been three major approaches to achieve the expansion of the genetic code: (i) Sense codon reassignment; (ii) Nonsense (stop) codon suppression; and (iii) non-triplet coding units (i.e. quadruplets). All three approaches make use of orthogonal tRNA (o-tRNA) and aminoacyl tRNA synthetase (o-aaRS)—the orthogonal pair.

Before the discovery and use of in vivo orthogonal aaRS/tRNA, the only practical way to incorporate UAAs into proteins relied on the use of chemically synthesized tRNAs synthetically aminoacylated to a UAA and added exogenously to the reaction mixture. These exogenous component and cell free methodologies were marked by the following limitations, among others as being laborious, time consuming and most importantly relied on stoichiometry as opposed to catalysis.

It was soon appreciated that instead of using synthetic tRNAs aminoacylated with UAA in vitro, exogenously added o-tRNA (Synthetic or Cell originated) and purified o-aaRS could be added to the reaction. Furthermore, use of partially recoded and RF1 deficient E. coli strains further increased suppression efficiency in these systems.

However, there are some major drawbacks in this methodology of cell free genetic expansion: Insoluble aaRS purification is very challenging—so challenging that Pyrrolysyl amino acyl tRNA synthetase (PylRS) and its derivatives, to the best of our knowledge, has never been purified in its full-length active form. As a consequence the cell free genetically expanded protein synthesis is limited to soluble aaRSs only, thus severely limiting the genetic expansion repertoire. Another major drawback is the labor and time needed for aaRS purification and tRNA synthesis, which if done correctly, take days and when the orthogonal pair is synthesized and purified it can only be stored in its active form for a relatively short time. Moreover, as these two essential components are synthesized and added exogenously it adds 2 more levels of complication and reduces the reproducibility and consistency of the results and products.

Thus, the drawbacks present a significant barrier to the widespread use of cell free genetically expanded protein.

Cell-free protein synthesis systems are a useful means to achieve accurate protein design and production, comparable to that attainable in living cells without the need for complicated post-translational purification steps.

There remains a need to have an industrially applicable synthesis system, improving synthesis efficiency; product stability and product yield in sufficient supply and high quality.

### SUMMARY OF THE INVENTION

In certain aspects of this invention, there is provided a cell-free protein synthesis system, which makes use of lysates from an E. coli expressed orthogonal pair from methanosarcina mazei (Mm): Mm-PylRS/tRNA_{cua}^{pyl} and derivatives thereof for preparing the cell free protein synthesis methods and kits as described herein.

In some embodiments, the pair is specifically introduced into a genomically recoded organism (GRO) C321:RF1−. In some embodiments, the pair is introduced in a non-genomically recoded organism. According to these aspects, it is noted that the methods/kits of this invention have been validated using at least 5 different E. coli strains as follows: BL21, DH5alpha, C321deltaPrfa, C321RF1+ and C321EXPdeltaPrfa).

In one aspect of this invention, the introduction of the orthogonal pair is prior to a cell lysis phase, and same results in the creation of an endogenous and all-inclusive lysate that will facilitate cell free stop codon (Amber or Ochre) suppression.

According to this aspect, and in some embodiments, such a cell free system will promote translation of the UAG triplet as a sense codon instead of a nonsense codon. In some aspects, the same enables cell-free protein synthesis to proceed without need for any addition of exogenous components (other than the UAA and the target gene to be expressed).

In some aspects, the systems of this invention provide minimal yields of 0.3 mg/ml.

As exemplified herein, when a destabilized eGFP Variant that has been optimized for CFPS (deGFP) was used as a model protein a proof of concept for enhanced cell free protein synthesis yields was demonstrated. As exemplified herein in Example 2, seamless expansion efficiency was demonstrated in all systems tested, including expression of an indicator compound and two active enzymes. Site-specific incorporation of the non-natural or rare amino acid was demonstrated in multiple sites, in various E. coli strains and using various orthogonal pairs.

Accordingly, this invention provides a method for producing a rare amino acid- or non-natural amino acid-containing protein in a cell free protein synthesis system said method comprising:
expressing at least one orthogonal suppressor tRNA (o-tRNA)/aminoacyl-tRNA synthetase (aaRS) pair or derivatives thereof specific for incorporation of a rare amino acid- or non-natural amino acid in an E. coli organism;
preparing a lysate of said E. coli organism expressing said orthogonal suppressor tRNA (o-tRNA)/aminoacyl-tRNA synthetase (aaRS) pair; and
contacting said lysate with a template DNA containing a mutant gene in which at least one amino acid codon at a given site of the protein-encoding gene has been mutated into an amber or ochre mutation and further providing a cognate rare amino acid or non-natural amino acid and other factors necessary for protein synthesis;
   wherein protein synthesis occurs following said contact to produce a protein containing said at least one rare amino acid or said non-natural amino acid.

In some aspects, the invention provides methods that make efficient use of the hard to purify pyrrolysyl-tRNA synthetase (PylRS) by expressing same in bacteria, prior to lysis. In some aspects, the invention provides methods that make efficient use of tyrosyl-tRNA synthetase by expressing same in bacteria, prior to lysis.

According to this aspect, the methods/kits provide for expressing an orthogonal suppressor tRNA (o-tRNA)/aminoacyl-tRNA synthetase (aaRS) pair (OTS) specific for incorporation of a rare amino acid- or non-natural amino acid in an E. coli organism, prior to lysis of same.

According to this aspect, the expression of the OTS prior to cell lysis promotes the easy and fast incorporation of any known UAA into the subsequently synthesized proteins and in some embodiments, such incorporation may be in one of many sites within the protein, or in some embodiments, within multiple sites in the protein.

Surprisingly, successful use of a PYL-OTS for site-specific incorporation of a UAA within a desired target protein was readily accomplished, as was successful use of an MJ-TyrRS family OTS, as well. The skilled artisan will therefore appreciate the applicability of the methods and kits of this invention for any appropriate OTS, in particular, for any TyrRS derivative, as well.

Surprisingly, and to our knowledge, for the first time, it was discovered possible to create a cell free protein synthesis system for incorporating two different UAAs in two different proteins, by preparing lysates comprising two distinct OTSs (Mj-Tyr and Mm-Pyl), respectively, and then by mixing the lysates together, facilitating the incorporation of the two different amino acids in two different sites.

Moreover, the successful incorporation of delta-thio-N-boc lysine using a cell free protein synthesis system was accomplished, providing a platform/method/kit for incorporation of this critically important UAA that can enable site specific ligation of two proteins together.

In some aspects, the method further comprises the step of producing two rare amino acid- or non-natural amino acid-containing proteins in a cell free protein synthesis system by synthesizing two proteins containing said at least one rare amino acid or said non-natural amino acid. According to this aspect, and in some embodiments, the method further comprises site-specific ligation of said two proteins.

In some embodiments of this invention, the method comprises expressing two different orthogonal suppressor tRNA (o-tRNA)/aminoacyl-tRNA synthetase (aaRS) pairs or derivatives thereof, specific for incorporation of two different cognate rare amino acids- or non-natural amino acids in an E. coli organism.

According to this aspect, and in some embodiments, one of the two rare or non-natural amino acids is p-azido-L-phenylalanine and said aminoacyl-tRNA synthetase is the tyrosyl-tRNA synthetase derivative Azido-L-Phenylalanine synthetase and the orthogonal tRNA is tRNA_{tyr}.

In some embodiments, according to this aspect, one of the two rare or non-natural amino acids is Propargyl-L-lysine and the aminoacyl-tRNA synthetase is pyrrolysyl-tRNA synthetase, and the orthogonal tRNA is tRNA_{pyl} or in some embodiments, one of the two rare or non-natural amino acids is N-Boc--Thio-L-lysine and the aminoacyl-tRNA synthetase is pyrrolysyl-tRNA synthetase and the orthogonal tRNA is tRNA_{pyl}. In other embodiments, according to this aspect, one of said two rare or non-natural amino acids is Δ-Thio-ε-Boc-Lysine and said aminoacyl-tRNA synthetase is pyrrolysyl-tRNA synthetase and the orthogonal tRNA is tRNA_{pyl}.

In other embodiments, this invention provides a kit for producing at least one rare amino acid- or non-natural amino acid-containing protein in a cell free protein synthesis system said kit comprising:
at least one E. coli lysate formed from an E. coli organism expressing at least one orthogonal suppressor tRNA (o-tRNA)/aminoacyl-tRNA synthetase (aaRS) pair specific for incorporation of a rare amino acid- or non-natural amino acid in said E. coli organism;
reaction mix comprising UTP, GTP, ATP, CTP, NAD, tRNAs, CoA, 3-PGA, cAMP, Folic Acid, K-Glutamate, Mg-Glutamate, Spermidine, natural amino acids, cognate rare amino acids or non-natural amino acids, crowding reagents, pH buffer, and combinations thereof; and
optionally at least one template DNA containing a mutant gene in which at least one amino acid codon at a given site of the protein-encoding gene has been mutated into an amber or ochre mutation.

In some embodiments, according to these aspects, the E. coli is genomically recoded to lack TAG codons in the genome and optionally to lack RF1.

In some aspects, the rare or non-natural amino acid utilized in the methods or kits of this invention is Propargyl-L-lysine and said aminoacyl-tRNA synthetase is pyrrolysyl-tRNA synthetase and the orthogonal tRNA is tRNA_{pyl}. In some embodiments, the rare or non-natural amino acid utilized in the methods or kits of this invention is N-Boc--Thio-L-lysine and said aminoacyl-tRNA synthetase is pyrrolysyl-tRNA synthetase the orthogonal tRNA is tRNA_{pyl}.

In some aspects, the rare or non-natural amino acid utilized in the methods or kits of this invention is p-azido-L-phenylalanine and said aminoacyl-tRNA synthetase is the tyrosyl-tRNA synthetase derivative Azido-L-Phenylalanine synthetase and the orthogonal tRNA is tRNA_{tyr}.

In some aspects, the rare amino acid utilized in the methods or kits of this invention is N-boc-L-lysine and said aminoacyl-tRNA synthetase is pyrrolysyl-tRNA synthetase and the orthogonal tRNA is tRNA_{pyl}.

In some aspects, the rare amino acid utilized in the methods or kits of this invention is Δ-Thio-ε-Boc-Lysine and said aminoacyl-tRNA synthetase is pyrrolysyl-tRNA synthetase and the orthogonal tRNA is tRNA_{pyl}.

In some embodiments of the methods of this invention and in use of the kits of this invention, the lysate is contacted with two different rare amino acids, which can be incorporated by the at least one orthogonal suppressor tRNA (o-tRNA)/aminoacyl-tRNA synthetase (aaRS) pair or derivatives thereof.

According to this aspect, and in some embodiments, the two different rare amino acids are Para-Azido-L-phenylalanine and Propargyl-L-lysine.

In some embodiments of the methods of this invention and in use of the kits of this invention, the lysate is contacted with two different rare or non-natural amino acids, which can be incorporated by the at least one orthogonal suppressor tRNA (o-tRNA)/aminoacyl-tRNA synthetase (aaRS) pair or derivatives thereof.

In some embodiments of the methods of this invention and in use of the kits of this invention, the protein containing at least one rare amino acid- or non-natural amino acid is a membrane-bound protein, or in some embodiments, the protein containing at least one rare amino acid- or non-natural amino acid is a secreted protein. In some embodiments of the methods of this invention and in use of the kits of this invention, the protein containing at least one rare amino acid- or non-natural amino acid is an enzyme, or in some embodiments, the protein containing at least one rare amino acid- or non-natural amino acid is an indicator protein.

In some embodiments of the methods of this invention and in use of the kits of this invention, the template DNA containing a mutant gene in which at least one amino acid codon at a given site of the protein-encoding gene has been mutated into an amber or ochre mutation is provided as a linear template, and in some embodiments, the template DNA containing a mutant gene in which at least one amino acid codon at a given site of the protein-encoding gene has been mutated into an amber or ochre mutation is provided within an expression plasmid.

In some embodiments of the methods and of the kits of this invention, there is provided template DNA containing a mutant gene in a reporter construct. In some embodiments the reporter construct facilitates quantitative assessment of protein synthesis efficiency using said kit.

In some embodiments of the methods and of the kits of this invention, there is provided a system and means of molecular sieving and in other embodiments of the methods and of the kits of this invention, there is provided continuous cell-free protein synthesis methods and kits for accomplishing same, which in some aspects, makes use of a dialysis membrane and relates to additional introduction of selected elements and appropriate apparatus therefor, as will be appreciated by the skilled artisan.

In some embodiments of the methods and of the kits of this invention, any mutant (derivative) of Methanomazei/Methanococcus barkeri Pyrrolysyl synthetase and/or of the Mj Tyrosine synthetase may be employed herein. According to this aspect, and in some embodiments, any of such mutant synthetases may be evolved to enable the incorporation of a different UAA.

According to this aspect, and in some embodiments, methods and of the kits of this invention which facilitate fabrication of different lysates, each containing a different synthetase (and a corresponding tRNA) allows for the broad incorporation of any UAA comprising the state of the art in the field.

### BRIEF DESCRIPTION OF THE DRAWINGS

FIG. 1 demonstrates Western Blot results showing successful incorporation of Propargyl-Lysine (UAA) site-specifically into proteins using a cell free protein synthesis (CFPS) system of this invention. 1a shows the incorporation of the UAA into distinct sites of deGFP. 1b shows the incorporation of the UAA into the 66^{th} amino acid site of Zymomonas mobilis Alcohol Dehydrogenase II (ADHII).

FIG. 2 plots the comparative stop codon suppression efficiencies between different E. coli strains assessed.

FIG. 3 plots the system stability and reproducibility, calculated as the standard deviation of independent reactions, most of them in different dates and with different batches of Extract, reaction Buffer and deGFP expression plasmid. Reaction parameters: Volume 10 ul, final Propargyl-Lysine (UAA) concentration 1 mM, expression plasmid concentration ˜4 nM. ANOVA test comparing between the W.T deGFP expression and both the Y35X deGFP (genetically expanded reaction) and the Y35X deGFP with no UAA added (negative control reaction) was calculated. Pval<0.0001 marked as ****

FIG. 4A and FIG. 4B verify the incorporation of PrK into deGFP by showing the deconvoluted ESI mass spectrum of purified WT deGFP and of purified deGFP Y35X (with incorporated PrK), respectively. FIG. 4C illustrates the potential for site-specific incorporation of PrK into deGFP and a sequential “Click” reaction to Tamra-azide fluorescent dye. FIG. 4D provides an image of an SDS-PAGE containing cell-free purified deGFP including PrK at position 35. Left lane contains Y35PrK deGFP after a “click” reaction with Tamra-azide, right lane contains un-reacted Y35PrK deGFP under the same experimental conditions. FIG. 4E plots detailed LC\MS data for WT deGFP (6×his). FIG. 4F shows the yields of genetically expanded CFPS reactions using a single batch of both lysate and buffer monitored over the course of time. FIG. 4G shows growth curves of the C321.ΔprfA strain and C321.ΔprfA cells transformed to express Pyl-OTS from plasmid pEVOL MmPylRS/MmPyltRNA, using varying arabinose concentrations. Each data point on the graph represents 10 sample repeats.

FIG. 5 plots activity of two proteins produced using the CFPS systems of this invention. 5a—E. coli copper efflux oxidase (CeuO). The activity CueO was measured by the OPD oxidation calorimetric assay. 5b Zymomonas mobilis Alcohol Dehydrogenase II (ADHII). The activity of the ADHII was measured by a colorimetric assay measuring NADH formation

FIG. 6 depicts the fate of the orthogonal pair OTS plasmid after lysis.

FIG. 7 schematically depicts an embodied cell free protein synthesis method of this invention.

FIG. 8 plots the results of EPI mass spectrometry for the deGFP variant containing TBL at position 35.

FIG. 9A plots the results for genetically expanded (Pyl-OTS) cell-free protein synthesis of deGFP. Expression kinetics of Y35X (where X is encoded by the TAG codon) deGFP, measured as fluorescence intensity, in the presence of varying concentrations of N^{ε}-Propargyl-l-lysine. FIG. 9B plots the results for genetically expanded (Pyl-OTS) cell-free protein synthesis of deGFP. Expression kinetics of Y35X (where X is encoded by the TAG codon) deGFP, measured as fluorescence intensity, in the presence of varying concentrations of N^{ε}-Boc-l-lysine.

FIG. 10A and FIG. 10B schematically depict embodied single and double extract CFPS systems of this invention, respectively.

FIG. 11A shows the results of an anti-GFP Western blot comparing the reaction results between wild type deGFP (i.e. deGFP lacking amber mutations) and Y35X deGFP with or without 1 mM of N^{ε}-Boc-l-lysine.

FIG. 11B plots the kinetics of Nε-Propargyl-l-lysine incorporation in multiple sites of deGFP using embodied CFPS systems, with the Pyl-OTS.

FIG. 12 and FIG. 13 plot the in vitro expression results of mixed lysate−C321 pEVOL pylRS TAG lysate+C321 pEVOL pAZF TAA lysate in terms of fluorescence. As a consequence of time the system achieves the incorporation of the two different UAAs in distinct sites. Various controls are presented.

FIG. 14 A and FIG. 14B provide the results for Western blot analysis probing using an anti-GFP antibody, validating the fluorescence measurement results in FIGS. 12 and 13, respectively.

FIG. 15A and FIG. 15B depict results of mass spectrometry for the Y35TAA D193TAG and Y35TAG D193TAA products, respectively.

FIG. 16 depicts the results of a click chemistry assay. A prominent band for each double mutant is seen in FIG. 16, as indicated, with only Lane 4 showing the absence of a band, since PrK was not provided in the cell free system for this sample.

FIG. 17A and FIG. 17B schematically depict fabrication of a ubiquitin code and ligation of the ubiquitin code to a substrate protein of interest, respectively.

FIG. 18 schematically depicts contemplated unnatural amino acids and rare amino acids for use in the embodied methods and kits as herein described.

### DETAILED DESCRIPTION OF THE INVENTION

This invention provides for a novel means of incorporating non-native amino acids into preselected positions of a protein using a cell-free synthesis system, including in some embodiments multiple positions within a single protein and in some embodiments, incorporation of different non-native amino acids into two different proteins.

This invention provides a cell-free protein synthesis system, which makes use of lysates from an E. coli which expressed an orthogonal pair suppressor tRNA (o-tRNA)/aminoacyl-tRNA synthetase (aaRS) pair or derivatives thereof specific for incorporation of a rare amino acid- or non-natural amino acid in an E. coli organism for preparing the cell free protein synthesis methods and kits as described herein.

In some aspects of the methods of this invention and making use of the kits of this invention, included in the methods are coupled transcription-translation reactions and in some aspects, the invention uses nonsense codon suppression during cell-free protein synthesis to produce high yields of polypeptides containing unnatural or rare amino acids.

In some aspects, the systems of this invention provide minimal yields of 0.3 mg/ml.

As exemplified herein, when a destabilized eGFP Variant that has been optimized for CFPS (deGFP) was used as a model protein a proof of concept for enhanced cell free protein synthesis yields was demonstrated. As exemplified herein in Example 2, seamless expansion efficiency was demonstrated in all systems tested, including expression of an indicator compound and two active enzymes. Site-specific incorporation of the non-natural or rare amino acid was demonstrated in multiple sites, in various E. coli strains and using various orthogonal pairs.

As further exemplified herein, an endogenously introduced orthogonal pair, enabled the use of the valuable yet insoluble pyrrolysyl tRNA synthetase in a cell-free system, and expansion of the genetic repertoire was validated using multiple UAAs (Examples 1-4), including incorporation of Δ-Thio-ε-Boc-Lysine (TBL), Propargyl-L-lysine, N-Boc--Thio-L-lysine and others. As further exemplified herein, use of single or mixed lysates provided a means for introducing different UAAs on a single protein and/or incorporation of two different UAAs in two different proteins, produced via a cell free protein synthesis platform and thereby providing for a variety of applications for use of same.

Accordingly, this invention provides a method for producing a rare amino acid- or non-natural amino acid-containing protein in a cell free protein synthesis system said method comprising:
expressing at least one orthogonal suppressor tRNA (o-tRNA)/aminoacyl-tRNA synthetase (aaRS) pair or derivatives thereof specific for incorporation of a rare amino acid- or non-natural amino acid in an E. coli organism;
preparing a lysate of said E. coli organism expressing said orthogonal suppressor tRNA (o-tRNA)/aminoacyl-tRNA synthetase (aaRS) pair; and
contacting said lysate with a template DNA containing a mutant gene in which at least one amino acid codon at a given site of the protein-encoding gene has been mutated into an amber or ochre mutation and further providing a cognate rare amino acid or non-natural amino acid and other factors necessary for protein synthesis;
wherein protein synthesis occurs following said contact to produce a protein containing said at least one rare amino acid or said non-natural amino acid.

This invention exploits the degeneracy of the genetic code to incorporate non-native amino acids into a growing polypeptide chain based on an mRNA sense codon sequence without compromising the ability to incorporate the native amino acid into the protein. Following cell lysis, a lysate is created that contains all the cellular components required for protein synthesis. A nucleic acid template is then added that has sense codons specifying positions in which the non-native amino acid will be incorporated.

In some embodiments of this invention, a target protein is synthesized in a cell-free reaction mixture comprising at least one orthogonal tRNA aminoacylated with an unnatural or rare amino acid, where the orthogonal tRNA base pairs with a nonsense codon that is not normally associated with an amino acid, e.g. a stop codon; a 4 bp codon, etc.

In some aspects, the terms “Aminoacylation” or “aminoacylate” or grammatical forms thereof refer to the complete process in which a tRNA is “charged” with its correct amino acid that is a result of adding an aminoacyl group to a compound. As it pertains to this invention, a tRNA that undergoes aminoacylation or has been aminoacylated is one that has been charged with an amino acid, and an amino acid that undergoes aminoacylation or has been aminoacylated is one that has been charged to a tRNA molecule.

In some aspects, the term “Aminoacyl-tRNA synthetase” or “tRNA synthetase” or “synthetase” or “aaRS” or “RS” refers to an enzyme that catalyzes a covalent linkage between an amino acid and a tRNA molecule. This results in a “charged” or “aminoacylated” tRNA molecule, which is a tRNA molecule that has its respective amino acid attached via an ester bond.

In some aspects, the term “Aminoacyl-tRNA synthetase” or “aaRS*” refers to mutant aminoacyl tRNA synthetase having enhanced specificity to non-natural amino acids. aaRS* thus defined can be obtained by introducing a mutation into a given site of known aminoacyl tRNA synthetase corresponding to natural amino acids. Known aminoacyl tRNA synthetase corresponding to natural amino acids first recognizes amino acids specifically, and it is activated with the addition of AMP, at the time of aminoacyl tRNA synthesis. Regarding known aminoacyl tRNA synthetase, a site that contributes to specific amino acid recognition is known, and such specificity can be changed by introducing a mutation into the relevant site. Based on such finding, a mutation that can reduce specificity to natural amino acids and enhance specificity to non-natural amino acids similar to the natural amino acids can be introduced. Thus, introduction of a mutation into a given site of known aminoacyl tRNA synthetase enables preparation of aaRS* having desired specificity.

Such aaRS* may be derived from prokaryotes, for example, mutant TyrRS, having enhanced specificity to 3-iodo-L-tyrosine (i.e., a non-natural amino acid), compared with specificity to tyrosine (i.e., a natural amino acid). Mutant TyrRS is described in the following document. (Kiga, D., Sakamoto, K., Kodama, K., Kigawa, T., Matsuda, T., Yabuki, T., Shirouzu, M., Harada, Y., Naklayama, H., Takio, K., Hasegawa, Y., Endo, Y., Hirao, I. and Yokoyama, S., 2002, An engineered Escherichia coli tyrosyl-tRNA synthetase for site-specific incorporation of an unnatural amino acid into proteins in eukaryotic translation and its application in a wheat germ cell-free system, Proc. Natl. Acad. Sci. U.S.A., 99, 9715-9723)

According to this document, substitution of sites corresponding to tyrosine (Y) at position 37 and glutamine (Q) at position 195 in E. coli-derived tyrosyl-tRNA synthetase with other amino acid residues enables production of mutants having enhanced specificity to 3-halogenated tyrosine (non-natural amino acids). In some embodiments, mutants in which a position corresponding to tyrosine (Y) at position 37 is substituted with valine (V), leucine (L), isoleucine (I), or alanine (A) and a position corresponding to glutamine (Q) at position 195 is substituted with alanine (A), cysteine (C), serine (S), or asparagine (N) can be used. Such mutants have particularly enhanced specificity to 3-iodo-L-tyrosine.

Genes encoding such mutants can be easily prepared by known genetic engineering techniques. For example, genes encoding such mutants can be obtained by site-directed mutagenesis or with the use of a commercialized kit for site-directed mutagenesis.

Examples of other aaRS* derived from prokaryotes include those described in Chin, J. W., Cropp, T. A., Anderson, J. C., Mukherji, M., Zhang, Z., and Schlutz, P. G., 2003, An expanded eukaryotic genetic code. Science, 301, 964-967 and those described in Deiters, A., Cropp, T. A., Mukherji, M., Chin, J. W., Anderson, J. C., and Schultz, P. G., 2003, Adding amino acids with novel reactivity to the genetic codes of Saccharomyces cerevisiae. J. Am. Chem. Soc. 125, 11782-11783. The skilled artisan will appreciate that the aaRS* envisioned for use in the methods and kits of this invention are in no way to be limited to those specified herein, but include any appropriate aaRS* known in the art.

The invention provides methods and kits which make use of at least one orthogonal suppressor tRNA (o-tRNA)/aminoacyl-tRNA synthetase (aaRS) pair or derivatives thereof specific for incorporation of a rare amino acid- or non-natural amino acid, which are expressed in a prokaryote, which in some embodiments, is in an E. coli organism.

In some embodiments, the term “suppressor tRNA” refers to a tRNA specific for non-natural amino acids. Such tRNA specific for non-natural amino acids may in turn be encoded by genes that encode tRNA, which are recognized by the aforementioned aaRS* and which have the 3′ terminus to which activated non-natural amino acids are transferred. Specifically, such aaRS* have activity of recognizing given non-natural amino acids, synthesizing non-natural amino acids-AMP, and transferring the non-natural amino acids to the 3′ terminus of tRNA for non-natural amino acids.

In some aspects, tRNA for non-natural, amino acids has an anticodon that is paired specifically with a genetic code other than the codons corresponding to 20 natural amino acid species. In some embodiments, an anticodon of tRNA for non-natural amino acids is composed of a sequence paired with a nonsense codon comprising an UAG amber codon, an UAA ochre codon, and an UGA opal codon.

In some embodiments, the aminoacyl-tRNA synthetase is pyrrolysyl-tRNA synthetase., or in some embodiments, the aminoacyl-tRNA synthetase is tyrosyl-tRNA synthetase or, in some embodiments, the aminoacyl-tRNA synthetase is the tyrosyl-tRNA synthetase derivative Azido-L-Phenylalanine synthetase.

In some aspects, the term “Lysate” is any cell derived preparation previously comprising the components required for protein synthesis machinery, wherein such cellular components are capable of expressing a nucleic acid encoding a desired protein. A lysate may be further combined with additional cellular components, as needed for cell free protein synthesis, including, e.g. amino acids, nucleic acids, enzymes, etc. The lysate may also be altered such that additional cellular components are removed following lysis.

The present invention provides a cell lysate prepared following in vivo translation of a target protein. For convenience, the organism used as a source for the lysate may be referred to as the source organism or host cell. Host cells may be bacteria, yeast, mammalian or plant cells, or any other type of cell capable of protein synthesis, and in particular, and as exemplified herein, the source organism is a prokaryote, which in some embodiments is an E. coli strain or derivative strain thereof.

In one embodiment, the methods and kits of this invention make use of a bacterial cell from which a lysate is derived. A bacterial lysate derived from any strain of bacteria can be used in the methods of the invention. The bacterial lysate can be obtained as follows. The bacteria of choice are grown up overnight in any of a number of growth media and under growth conditions that are well known in the art and easily optimized by a practitioner for growth of the particular bacteria. For example, a natural environment for synthesis utilizes cell lysates derived from bacterial cells grown in medium containing glucose and phosphate, where the glucose is present at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients. Cells that have been harvested overnight can be lysed by suspending the cell pellet in a suitable cell suspension buffer, and disrupting the suspended cells by sonication, breaking the suspended cells in a French press, continuous flow high pressure homogenization, or any other method known in the art useful for efficient cell lysis.

In some aspects, the term “Non-native amino acids” or “nnAA” refer to amino acids that are not one of the twenty naturally occurring amino acids that are the building blocks for all proteins that are nonetheless capable of being biologically engineered such that they are incorporated into proteins. In some embodiments, such nnAA are also referred to herein as unnatural amino acids or “UAA”, or in some embodiments, rare amino acids.

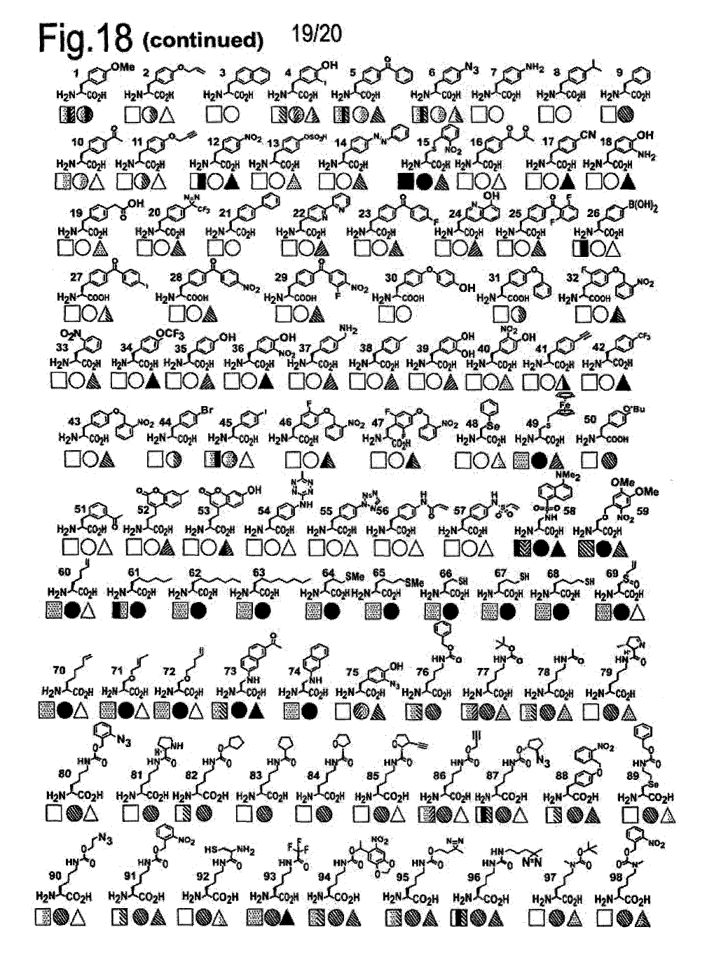

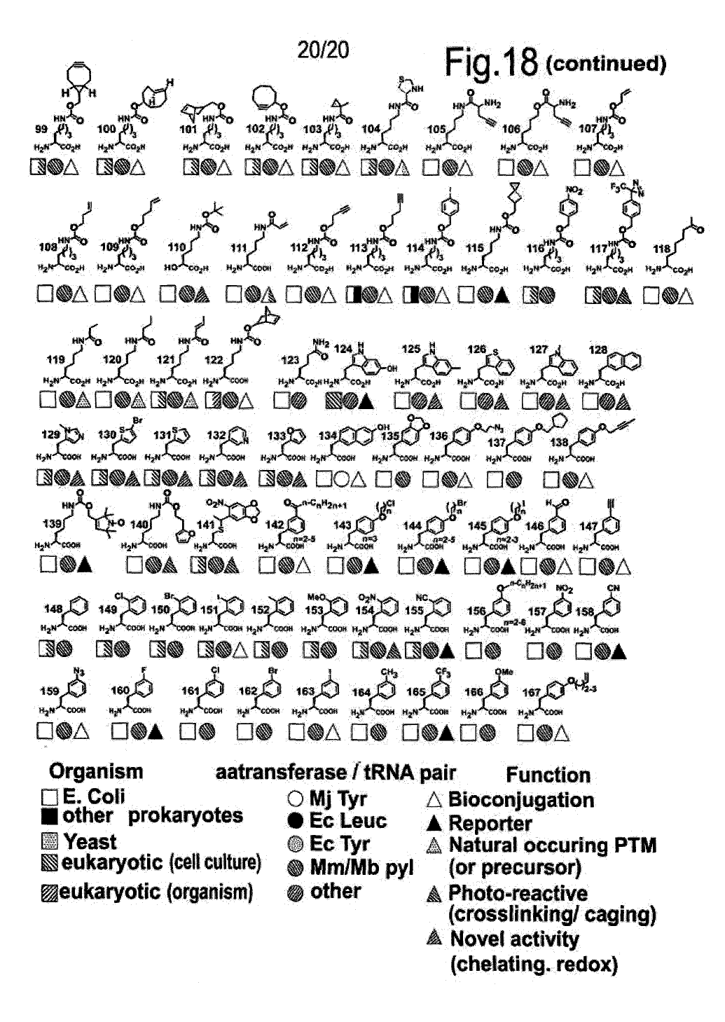

Non-native amino acids may include D-peptide enantiomers or any post-translational modifications of one of the twenty naturally occurring amino acids. A wide variety of non-native amino acids can be used in the methods of the invention. The non-native amino acid can be chosen based on desired characteristics of the non-native amino acid, e.g., function of the non-native amino acid, such as modifying protein biological properties such as toxicity, biodistribution, or half-life, structural properties, spectroscopic properties, chemical and/or photochemical properties, catalytic properties, ability to react with other molecules (either covalently or noncovalently), or the like. Non-native amino acids that can be used in the methods of the invention may include, but are not limited to, an non-native analogue of a tyrosine amino acid; an non-native analog of a glutamine amino acid; an non-native analog of a phenylalanine amino acid; an non-native analog of a serine amino acid; an non-native analog of a threonine amino acid; an alkyl, aryl, acyl, azido, cyano, halo, hydrazine, hydrazide, hydroxyl, alkenyl, alkynl, ether, thiol, sulfonly, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analog containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; a cyclic amino acid other than praline, etc. In some embodiments, the unnatural or rare amino acids as described herein may include any amino acid as described in FIG. 18.

In some embodiments, the rare or non-natural amino acid is Propargyl-L-lysine, or in some embodiments, the rare or non-natural amino acid is N-Boc--Thio-L-lysine, or in some embodiments, the rare or non-natural amino acid is p-azido-L-phenylalanine, or in some embodiments, the rare or non-natural amino acid is N-boc-L-lysine, or in some embodiments, the rare or non-natural amino acid is Δ-Thio-ε-Boc-Lysine.

Unnatural or rare amino acids constitute any amino acid analog or similar entity that is not commonly found in nature, including but not limited to those molecules that can be used for targeted post-translational modification. The reaction mixture comprises cell extracts, which are optionally amino acid stabilized, reductase minimized, and/or protease mutated cell extracts.

In order to produce the proteins of this invention, one needs a nucleic acid template. The template for cell-free protein synthesis can be either mRNA or DNA. The template can encode for any particular gene of interest, and may encode a full-length polypeptide or a fragment of any length thereof. Nucleic acids to serve as sequencing templates are optionally derived from a natural source or they can be synthetic or recombinant. For example, DNAs can be recombinant DNAs, e.g., plasmids, viruses or the like.

A DNA template that comprises the gene of interest will be operably linked to at least one promoter and to one or more other regulatory sequences including without limitation repressors, activators, transcription and translation enhancers, DNA-binding proteins, etc. Suitable quantities of DNA template for use herein can be produced by amplifying the DNA in well-known cloning vectors and hosts, or by polymerase chain reaction (PCR).

A preferred embodiment uses a bacterial lysate. A DNA template may be constructed for bacterial expression by operably linking a desired protein-encoding DNA to both a promoter sequence and a bacterial ribosome binding site (Shine-Delgarno sequence). Promoters suitable for use with the DNA template in the cell-free transcription-translation methods of the invention include any DNA sequence capable of promoting transcription in vivo in the bacteria from which the bacterial extract is derived. Preferred are promoters that are capable of efficient initiation of transcription within the host cell. DNA encoding the desired protein and DNA containing the desired promoter and Shine-Dalgarno (SD) sequences can be prepared by a variety of methods known in the art. Alternatively, the desired DNA sequences can be obtained from existing clones or, if none are available, by screening DNA libraries and constructing the desired DNA sequences from the library clones.

RNA templates encoding the protein of interest can be conveniently produced from a recombinant host cell transformed with a vector constructed to express an mRNA with a bacterial ribosome binding site (SD sequence) operably linked to the coding sequence of the desired gene such that the ribosomes in the reaction mixture are capable of binding to and translating such mRNA. Thus, the vector carries any promoter capable of promoting the transcription of DNA in the particular host cell used for RNA template synthesis.

Examples of appropriate molecular techniques for generating recombinant nucleic acids, and instructions sufficient to direct persons of skill through many closing exercises are found in Berger and Kimmel, Guide to Molecular Cloning Techniques, Methods in Enzymology (Volume 152 Academic Press, Inc., San Diego, Calif. 1987); PCR Protocols: A Guide to Methods and Applications (Academic Press, San Diego, Calif. 1990). Product information from manufacturers of biological reagents and experimental equipment also provide information useful in known biological methods. Such manufacturers include SIGMA (Saint Louis, Mo.), R&D systems (Minneapolis, Minn.), Pharmacia LKB Biotechnology (Piscataway, N.J.), Clontech Laboratories, Inc. (Palo Alto, Calif.), Aldrich Chemical Company (Milwaukee, Wis.), Invitrogen (San Diego, Calif.), Applied Biosystems (Fosters City, Calif.), as well as many other commercial sources known to one of skill in the art.

This invention, in some aspects, specifically relates to methods and kits for cell free protein synthesis applications.

The term “cell-free protein synthesis” refers to synthesis of a target protein by adding nucleic acid template for a gene encoding such protein.

As part of the methods and kits of this invention, a lysate is utilized for effecting cell-free protein synthesis. In some embodiments, such lysate is an extract obtained by isolating prokaryotic cells, which in some embodiments, are typified by E. coli, and other related strains and forming a lysate thereof via conventional techniques. In some aspects, insoluble substances are removed via centrifugation or other means. In some aspects, endogenous DNA and RNA are degraded by a conventional technique, and endogenous amino acids, nucleic acids, nucleosides, or the like are removed or a pH level and a salt concentration is adjusted via dialysis of other means, according to need. The obtained lysate, according to this aspect, retains the ability of protein synthesis including ribosome.

In some embodiments, an E. coli lysate can be prepared in accordance with the method described in, for example, Pratt, J. M. et al., Transcription and Translation, Hames, 179-209, B. D. & Higgins, S. J., eds., IRL Press, Oxford, 1984, or as exemplified herein, or as described elsewhere. Methods for preparing an extract from cells are not limited to those described above, and any methods can be employed.

In some embodiments, after the lysate is prepared, ingredients necessary for protein synthesis can be added in order to prepare a solution for cell-free protein synthesis. Ingredients necessary for protein synthesis may be stored separately from the lysate, and in some embodiments, kits as disclosed herein are particularly useful for effecting the methods of this invention. In some aspects, the kits may comprise the lysate alone, and in some embodiments, the kits may optionally comprise other ingredients as needed.

In some embodiments, such ingredients may be mixed with the lysate at the time of use. Ingredients necessary for protein synthesis are not particularly limited. Examples thereof include Tris-acetic acid, DTT, NTPs (ATP, CTP, GTP, and UTP), RNA polymerase, phosphoenolpyruvic acid, pyruvate kinase, at least one type of amino acid (including 20 types of naturally-occurring amino acids and derivatives thereof), polyethylene glycol (PEG), folic acid, cAMP, tRNA, ammonium acetate, potassium acetate, potassium glutamate, and magnesium acetate at the optimal concentration, in addition to the unnatural or rare amino acids as described herein and at least one orthogonal suppressor tRNA (o-tRNA)/aminoacyl-tRNA synthetase (aaRS) pair or derivatives thereof specific for incorporation of a rare amino acid- or non-natural amino acid in an E. coli organism.

In some aspects, the methods of this invention relating to the preparation of the cell lysate as herein described may further comprise a freeze-thawing procedure, or in some embodiments, the methods and kits of this invention make use of an E. coli strain in which a mutation is introduced into the me gene encoding an endonuclease RNase E. in some embodiments, the methods and kits of this invention make use of various mutant E. coli strains which are RecBCD deficient.

In some aspects, the invention provides methods that make efficient use of the hard to purify pyrrolysyl-tRNA synthetase (PylRS) by expressing same in bacteria, prior to lysis. In some aspects, the invention provides methods that make efficient use of tyrosyl-tRNA synthetase by expressing same in bacteria, prior to lysis.

According to this aspect, the methods/kits provide for expressing an orthogonal suppressor tRNA (o-tRNA)/aminoacyl-tRNA synthetase (aaRS) pair (OTS) specific for incorporation of a rare amino acid- or non-natural amino acid in an E. coli organism, prior to lysis of same.

According to this aspect, the expression of the OTS prior to cell lysis promotes the easy and fast incorporation of any known UAA into the subsequently synthesized proteins and in some embodiments, such incorporation may be in one of many sites within the protein, or in some embodiments, within multiple sites in the protein.

Surprisingly, successful use of a PYL-OTS for site-specific incorporation of a UAA within a desired target protein was readily accomplished, as was successful use of an MJ-TyrRS family OTS, as well. The skilled artisan will therefore appreciate the applicability of the methods and kits of this invention for any appropriate OTS, in particular, for any TyrRS derivative, as well.

Surprisingly, and to our knowledge, for the first time, it was discovered possible to create a cell free protein synthesis system for incorporating two different UAAs in two different proteins, by preparing lysates comprising two distinct OTSs (Mj-Tyr and Mm-Pyl), respectively, and then by mixing the lysates together, facilitating the incorporation of the two different amino acids in two different sites.

Moreover, the successful incorporation of delta-thio-N-boc lysine using a cell free protein synthesis system was accomplished, providing a platform/method/kit for incorporation of this critically important UAA that can enable site specific ligation of two proteins together. In some embodiments, the site-specific ligation of two proteins may, for example, include applications of native chemical ligation reactions to create an iso peptide bond, or in some embodiments, other chemical reactions may be effected and the skilled artisan will appreciate that the same should not be limited and is a contemplated embodiment of this invention.

In some aspects, the method further comprises the step of producing two rare amino acid- or non-natural amino acid-containing proteins in a cell free protein synthesis system by synthesizing two proteins containing said at least one rare amino acid or said non-natural amino acid. According to this aspect, and in some embodiments, the method further comprises site-specific ligation of said two proteins.

In some aspects, the lysate is contacted with two different rare amino acids, which can be incorporated by the at least one orthogonal suppressor tRNA (o-tRNA)/aminoacyl-tRNA synthetase (aaRS) pair or derivatives thereof. According to this aspect, and in some embodiments, the two different rare amino acids are Para-Azido-L-phenylalanine and Propargyl-L-lysine.

In some embodiments of this invention, the method comprises expressing two different orthogonal suppressor tRNA (o-tRNA)/aminoacyl-tRNA synthetase (aaRS) pairs or derivatives thereof, specific for incorporation of two different cognate rare amino acids- or non-natural amino acids in an E. coli organism.

According to this aspect, and in some embodiments, one of the two rare or non-natural amino acids is p-azido-L-phenylalanine and said aminoacyl-tRNA synthetase is the tyrosyl-tRNA synthetase derivative Azido-L-Phenylalanine synthetase.

In some embodiments, according to this aspect, one of the two rare or non-natural amino acids is Propargyl-L-lysine and the aminoacyl-tRNA synthetase is pyrrolysyl-tRNA synthetase, or in some embodiments, one of the two rare or non-natural amino acids is N-Boc--Thio-L-lysine and the aminoacyl-tRNA synthetase is pyrrolysyl-tRNA synthetase. In other embodiments, according to this aspect, one of said two rare or non-natural amino acids is Δ-Thio-ε-Boc-Lysine and said aminoacyl-tRNA synthetase is pyrrolysyl-tRNA synthetase.

The methods and kits of this invention provide for the incorporation of a rare amino acid- or non-natural amino acid-containing protein in a cell free protein synthesis system, where in some embodiments, such protein containing at least one rare amino acid- or non-natural amino acid is a membrane-bound protein, or in some embodiments, such protein containing at least one rare amino acid- or non-natural amino acid is a secreted protein, or in some embodiments, such protein containing at least one rare amino acid- or non-natural amino acid is an enzyme, or in some embodiments, such protein containing at least one rare amino acid- or non-natural amino acid is an indicator protein.

In some embodiments, other proteins or polypeptides of interest containing at least one rare amino acid- or non-natural amino acid by the methods and/or making use of the kits of this invention include, without limitation, proteins containing disulfide bonds, any heterogenous or homogeneous combination of proteins, including fusion proteins, viral coat proteins, and/or proteins originally secreted through or within a cellular membrane.

In some embodiments, enzymes and other reporter proteins are contemplated. In one embodiment, insulin is specifically contemplated. In another embodiment, ubiquitin is specifically contemplated.

Although orthogonal tRNA can be reliably synthesized by bacterial cells from which an extract for cell-free synthesis is made, the orthogonal tRNA synthetase has been found to be susceptible to degradation in the bacterial cell extracts, or to competition with endogenous release factor 1 (RF1). tRNA is susceptible to degradation, as well, both in the extracts, and in purified form, for example, during storage

Surprisingly, it is now found that both orthogonal tRNA and orthogonal tRNA synthetase can be reliably synthesized by bacterial cells from which an extract for cell-free synthesis is subsequently made by making use of specifically mutated or genetically recoded organisms, for example, by using an E. coli organism genomically recoded to lack TAG codons in the genome and optionally to lack RF1.

In some aspects, the methods of the invention provide for high yields of active, modified protein, which may be greater than the yield that can be achieved with in vivo expression systems. In one embodiment of the invention, the yield of active modified protein is at least about 50 μg/ml of reaction mixture; at least about 100 μg/ml of reaction mixture; at least about 250 μg/ml of reaction mixture; or more. A substantial portion of the target polypeptide thus produced contains the desired unnatural or rare amino acid, which in some embodiments is at least about 50%, at least about 75%, at least about 85%, at least about 95%, at least about 99%, or higher.

A modified protein, or target protein, as used herein, comprises at least one unnatural or rare amino acid at a pre-determined site, and may comprise or contain 1, 2, 3, 4, 5 or more unnatural or rare amino acids. If present at two or more sites in the polypeptide, the unnatural or rare amino acids can be the same or different.

Where the unnatural or rare amino acids are different, an orthogonal tRNA and cognate tRNA synthetase will be expressed in the same or different plasmids, in an E. coli organism genomically recoded to lack RF1, for each unnatural or rare amino acid.

In some aspects, the methods of the present invention provide for proteins containing unnatural or rare amino acids that have biological activity comparable to the native protein.

In some aspects, one may determine the specific activity of a protein in a composition by determining the level of activity in a functional assay, quantitating the amount of protein present in a non-functional assay, e.g. Western Blot analysis, immunostaining, ELISA, quantitation on coomasie or silver stained gel, etc., and determining the ratio of biologically active protein to total protein.

In some embodiments, the specific activity as thus defined will be at least about 5% that of the native protein, usually at least about 10% that of the native protein, and may be about 25%, about 50%, about 90% or greater.

Preparation of cell extracts for cell-free protein synthesis of the present invention from these raw material cells may be performed in combination with various known methods (Johnston, F. B. et al. (1957) Nature, 179, 160-161). In some embodiments, the raw material cells are also treated with a surfactant, including in some embodiments, a nonionic surfactant.

A wide variety of nonionic surfactants may be used such as, for example, Brij, Triton, Nonidet P-40, Tween, and the like, which are polyoxyethylene derivatives. In some embodiments, the nonionic surfactants are used in a concentration of, for example, 0.5%.

Traditionally, storing the cell extracts for cell-free protein synthesis is at temperatures in the vicinity of −80 to −196° C.

In some aspects, the cell extracts for cell-free protein synthesis as described herein are formed by means of a dry process, such as freeze-drying.

To the cell free synthesis systems may be added substances which increase the reaction efficiency, as will be appreciated by the skilled artisan. In some embodiments, for example, various ionic compounds, such as potassium ion compound, magnesium ion compound, etc. may be added.

Further, to the preparation may, if desired, be added substances which enhances solubility, for example, surfactants, substances which protects the above ribosomes from deadenylation thereof and others, as will be appreciated by the skilled artisan.

In some aspects, template DNA or mRNA serving as a template for a synthesis reaction is supplemented on demand or continuously to the extracts as herein defined. The addition may be made on demand in a very small amount continually, or periodically.

In the present invention, an enzyme in an energy reproduction system may be supplemented on demand or continuously after initiating the reaction. The addition may be made in a very small amount continually or periodically.

The supplemental additions of mRNA and the enzyme for the energy reproduction system may be performed separately from each other, or in combination in other embodiments. The addition method may be either continuously or intermittently.

In some aspects, there is also contemplated a step for preventing the exhaustion of substrate and/or, energy source and/or a step for discharging by-products.

In some aspects, various amino acids, ATP, GTP, etc. are supplementally added as a substrate or energy source continuously or intermittently. Such addition amounts may be supplemented or changed when needed.

In some embodiments, discharge of the by-products for example, discharging metabolites such as AMP and GMP, etc., and reaction products, such as phosphoric acid and pyrophosphoric acid, etc., and such compounds from the reaction system continuously or intermittently is contemplated.

In some embodiments, steps for preventing the exhaustion of substrate and/or energy source, and/or steps for discharging by-products are/is preferably continuous or intermittent renewal of the reaction medium in the reaction system are contemplated. In some embodiments, for example, the method may make further use of a dialysis membrane.

The uses of proteins containing non-native amino acids include desired changes in protein structure and/or function, which would include changing the size, acidity, nucleophilicity, hydrogen bonding, hydrophobicity, or accessibility of protease target sites. Proteins that include an non-native amino acid can have enhanced or even entirely new catalytic or physical properties such as modified toxicity, biodistribution, structural properties, spectroscopic properties, chemical and/or photochemical properties, catalytic ability, serum half-life, and the ability to react with other molecules, either covalently or noncovalently. Proteins that include at least one non-native amino acid are useful for, but not limited to, novel therapeutics, diagnostics, catalytic enzymes, binding proteins, and the study of protein structure and function.

The modified protein may also be referred to as the desired protein, selected protein, or target protein. In some embodiments, the modified protein refers generally to any peptide or protein having more than about 5 amino acids. The modified protein comprises at least one non-native amino acid at a pre-determined site, and may contain multiple non-native amino acids. If present at two or more sites in the polypeptide, the non-native amino acids can be the same or different. Where the non-native amino acids are different, the tRNA codons for each non-native amino acids will also be different.

The modified protein may be homologous to, or may be exogenous, meaning that they are heterologous, i.e., foreign, to the cells from which the lysate is derived, such as a human protein, viral protein, yeast protein, etc. produced in a bacterial cell-free extract. Modified proteins may include, but are not limited to, molecules such as, e.g., renin, a growth hormone, including human growth hormone; bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-(3; platelet-derived growth factor (PDGF); fibroblast growth factor such as aFGF and bFGF; epidermal growth factor (EOF); transforming growth factor (TGF) such as TGF-alpha and TGF-beta, including TGF-(31, TGF-(32, TGF-(33, TGF-(34, or TGF-(35; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-I 9; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; antibodies; and fragments of any of the above-listed polypeptides.

The target proteins incorporating the UAA or rare amino acid can, in some embodiments, be used for (i) structure determination via X-ray crystallographic analysis, (ii) photo-crosslinking or site-directed fluorescent labeling for elucidation of cell signaling pathways, (iii) use as a proteinous drug upon site-directed polyethyleneglycolation for enhancing drug efficacy, and other purposes. According to protein function analysis via site-directed amino acid substitution, amino acids that can be used for substitution are limited to 20 natural amino acid species in the past. Use of non-natural amino acids enables amino acid substitution with a wide variety of amino acid residues without limitation. Thus, analysis of prepared mutants enables elucidation of roles of amino acid residues at specific sites in proteins, as well.

In some embodiments, the methods and kits of this invention are suitable for automation.

This invention also provides a kit for producing at least one rare amino acid- or non-natural amino acid-containing protein in a cell free protein synthesis system said kit comprising:
at least one E. coli lysate formed from an E. coli organism expressing at least one orthogonal suppressor tRNA (o-tRNA)/aminoacyl-tRNA synthetase (aaRS) pair specific for incorporation of a rare amino acid- or non-natural amino acid in said E. coli organism;
reaction mix comprising UTP, GTP, ATP, CTP, NAD, tRNAs, natural amino acids, cognate rare amino acids or non-natural amino acids, crowding reagents, pH buffer, and combinations thereof; and
optionally at least one template DNA containing a mutant gene in which at least one amino acid codon at a given site of the protein-encoding gene has been mutated into an amber or ochre mutation.

In some embodiments, the methods and kits of this invention make use of any appropriate cell, and in some embodiments, the methods and kits of this invention make use of any appropriate prokaryote, and in some embodiments, the methods and kits of this invention make use of any appropriate bacterial strain, including E. coli and derivative strains thereof and lysates are prepared from same, as described hereinabove, and are to be considered contemplated as part of the lysates suitable for inclusion in the kits of this invention.

In some embodiments, the pH buffer for use in the methods and/or kits of this invention is any suitable buffer, for example, HEPES buffer.

In some embodiments, according to these aspects, the E. coli is any genomically recoded to lack TAG codons in the genome and optionally to lack RF1.

In some aspects, the rare or non-natural amino acid utilized in the kits of this invention is Propargyl-L-lysine and said aminoacyl-tRNA synthetase is pyrrolysyl-tRNA synthetase. In some embodiments, the rare or non-natural amino acid utilized in the methods or kits of this invention is N-Boc--Thio-L-lysine and said aminoacyl-tRNA synthetase is pyrrolysyl-tRNA synthetase.

In some aspects, the rare or non-natural amino acid utilized in the kits of this invention is p-azido-L-phenylalanine and said aminoacyl-tRNA synthetase is the tyrosyl-tRNA synthetase derivative Azido-L-Phenylalanine synthetase, or any embodied rare or non-natural amino acid described hereinabove.

In some aspects, the rare amino acid utilized in the kits of this invention is N-boc-L-lysine and said aminoacyl-tRNA synthetase is pyrrolysyl-tRNA synthetase.

In some aspects, the rare amino acid utilized in the kits of this invention is Δ-Thio-ε-Boc-Lysine and said aminoacyl-tRNA synthetase is pyrrolysyl-tRNA synthetase.

In some embodiments of the kits of this invention, the lysate is contacted with two different rare amino acids, which can be incorporated by the at least one orthogonal suppressor tRNA (o-tRNA)/aminoacyl-tRNA synthetase (aaRS) pair or derivatives thereof and the appropriate factors for same (for example, one or two, or more orthogonal suppressor tRNA (o-tRNA)/aminoacyl-tRNA synthetase (aaRS) pair or derivatives thereof and one or two or more are included within the kits of this invention. According to this aspect, and in some embodiments, the two different rare amino acids are Para-Azido-L-phenylalanine and Propargyl-L-lysine.

In some embodiments of the kits of this invention, the lysate is contacted with two different rare or non-natural amino acids, which can be incorporated by the at least one orthogonal suppressor tRNA (o-tRNA)/aminoacyl-tRNA synthetase (aaRS) pair or derivatives thereof.

In some embodiments of the kits of this invention, the protein containing at least one rare amino acid- or non-natural amino acid is a membrane-bound protein, or in some embodiments, the protein containing at least one rare amino acid- or non-natural amino acid is a secreted protein. In some embodiments of the methods of this invention and in use of the kits of this invention, the protein containing at least one rare amino acid- or non-natural amino acid is an enzyme, or in some embodiments, the protein containing at least one rare amino acid- or non-natural amino acid is an indicator protein.

In some embodiments of the kits of this invention, the template DNA containing a mutant gene in which at least one amino acid codon at a given site of the protein-encoding gene has been mutated into an amber or ochre mutation is provided as a linear template, and in some embodiments, the template DNA containing a mutant gene in which at least one amino acid codon at a given site of the protein-encoding gene has been mutated into an amber or ochre mutation is provided within an expression plasmid.

In some embodiments of the kits of this invention, there is provided template DNA containing a mutant gene in a reporter construct. In some embodiments the reporter construct facilitates quantitative assessment of protein synthesis efficiency using said kit.

In some embodiments of the methods and of the kits of this invention, there is provided a system and means of molecular sieving and in other embodiments of the methods and of the kits of this invention, there is provided continuous cell-free protein synthesis methods and kits for accomplishing same, which in some aspects, makes use of a dialysis membrane and relates to additional introduction of selected elements and appropriate apparatus therefor, as will be appreciated by the skilled artisan.

In some embodiments of the methods and of the kits of this invention, any mutant (derivative) of Methanomazei/Methanococcus barkeri Pyrrolysyl synthetase and/or of the Mj Tyrosine synthetase may be employed herein. According to this aspect, and in some embodiments, any of such mutant synthetases may be evolved to enable the incorporation of a different UAA.

According to this aspect, and in some embodiments, methods and of the kits of this invention which facilitate fabrication of different lysates, each containing a different synthetase (and a corresponding tRNA) allows for the broad incorporation of any UAA comprising the state of the art in the field.

### EXAMPLES

### Materials & Methods

For the cloning of genetic expansion of orthogonal pair systems (OTS) into plasmids containing orthogonal pairs and transforming into E. coli strains, the following plasmids were used: pEVOL-PylRS (as described in Blight, Sherry K., et al. “Direct charging of tRNACUA with pyrrolysine in vitro and in vivo.” Nature 431.7006 (2004): 333-335), pEVOLPylRS-AF (as described in Plass, Tilman, et al. “Genetically Encoded Copper-Free Click Chemistry.” Angewandte Chemie International Edition 50.17 (2011): 3878-3881), pSUPpACF (as described in Ryu, Youngha, and Peter G. Schultz. “Efficient incorporation of unnatural amino acids into proteins in Escherichia coli.” Nature methods (2006): 263-265), pKDSepRS (as described in Park, Hee-Sung, et al. “Expanding the genetic code of Escherichia coli with phosphoserine.” Science 333.6046 (2011): 1151-1154) and no OTS vector. Strains of used E. coli used are C321.ΔPrfA, C321.ΔPrfAEXP, C321RF1+(purchased from addgene under MTA from Lajoie, Marc J., et al. “Genomically recoded organisms expand biological functions.” science 342.6156 (2013): 357-360.), BL21/DH5α (New England biolabs Inc.) and Rosetta (Merck-Millipore Co.) All the plasmids were introduced into the C321.ΔPrfA strain, and pEVOLPylRS was introduced into each E. coli strain. Bacterial strains and plasmids used are as also further described in the tables provided below.

Following transformation of the indicated strains with the respective plasmids in accordance with the schedule in Table 1 below, cells were permitted to go through a growth and induction phase, whereby the elements of the orthogonal pair system, i.e. the orthogonal tRNA (o-tRNA) and orthogonal Aminoacyl tRNA synthetase (o-aaRS) accumulate at expanded levels within the cells. Lysates of the genetically expanded cells were prepared.

All transformations of the C321.ΔprfA, C321.RF1+, BL21, DH5α & Rosetta II strains were done by electroporation. Parent strains not containing any plasmids were grown in Luria-Bertani (LB) broth (10 g/L NaCl, 10 g/L trypton and 5 g/L yeast extract) overnight at 30° C. (C321 derivatives) or 37° C. (BL21 & Rosetta) for sequential inoculation. The cultures were diluted 1/100 in fresh LB, and incubated at the relevant temperature while being shaken at 275 rpm to OD600 of 0.5-0.7. Cells were then harvested and washed with 10% glycerol in water three times, aliquoted and stored at −80° C. until thawed for transformation. Cells were exposed to DNA using 50-100 ng/μL of template DNA (obtained by mini-prep) and then transformed by electroporation using a MictroPulser (Bio-Rad). Transformed cells were then incubated for 1-1.5 h in SOC broth (2% bacto-tryptone, 0.5% bacto-yeast extract, 10 mM NaCl, 2.5 mM KCl, 10 mM MgCl2, 10 mM MgSO4 and 20 mM glucose) at the appropriate temperature and sequentially plated on selective LB-agar plates containing the proper antibiotic. Competent DH5-alpha E. coli cells (New England Biolabs) were transformed using the prescribed heat shock protocol and sequentially plated on selective LB-agar plates.

Site directed Mutagenesis introduced a TAG codon at desired sites in the following genes: a destabilized eGFP variant that undergoes degradation (deGFP), the zibomonas mobilis alcohol dehydrogenase (ADH) gene and the E. coli Copper efflux oxidase (CueO) genes, for subsequent UAA incorporation, serving as the target genes, as indicated. The ZmADH gene was mutated to contain a V66TAG mutation and subcloned to the pBEST plasmid for sequential expression and UAA incorporation in the CFPS system. The Ecoli CueO was mutated to contain a H117TAG mutation and subcloned to the pBest plasmid for sequential expression and UAA incorporation in the CFPS system. Both mutation in both plasmid allow the incorporation of all UAAs given the correct orthogonal translational system (OTS) is present in the reaction lysate. This is achieved though amber suppression. In our invention we have showcased this by the incorporation of propargyl-lysine by the pylRS/PylT orthogonal pair. Toward this end, primers were designed and PCR-mediated mutagenesis was conducted according to Ho, Steffan N., et al. “Site-directed mutagenesis by overlap extension using the polymerase chain reaction.” Gene 77.1 (1989): 51-59. Once the TAG mutation was introduced, template DNA containing the respective target gene with the site specific amber mutation was prepared.

Cell extract preparation. The cell extract (crude extract) was prepared as described, with some modifications (Sun, Z. Z., Hayes, C. a, Shin, J., Caschera, F., Murray, R. M., and Noireaux, V. (2013) Protocols for implementing an escherichia coli based TX-TL cell-free expression system for synthetic biology. J. Vis. Exp. 79, 1-15; Liu, D. V, Zawada, J. F., and Swartz, J. R. (2005) Streamlining Escherichia coli S30 extract preparation for economical cell-free protein synthesis. Biotechnol. Prog. 21, 460-465; Kigawa, T., Yabuki, T., Matsuda, N., Matsuda, T., Nakajima, R., Tanaka, A., and Yokoyama, S. (2004) Preparation of Escherichia coli cell extract for highly productive cell-free protein expression. J. Struct. Funct. Genomics 5, 63-68). Selection antibiotics were adjusted (according to the transforming plasmid used) and the temperature of growth, which influenced the growth incubation times of starter cultures (i.e. cultures that are diluted to grow the harvested culture); i.e. in order to have the different strains experience the same number of generations. For example, a strain with a doubling time of ˜30 minutes was incubated for ˜8 h, whereas a strain with a doubling time of 50 min was incubated for ˜13 h. Additionally, the promoter regulating the expression of the aaRS was induced in early log phase (OD600 0.5-0.7) (for plasmid pEVOL, 0.5-1% L-arabinose was added, while for plasmid pKD, 1 mM IPTG was added; nothing was added for plasmid pSUP that lacks an inducible promoter) of growth, resulting in over-expression of the aaRS, thus enabling cell-free o-tRNA amino-acetylation once exogenous UAA is introduced. S30A buffer comprises 14 mM Mg-glutamate, 60 mM K-glutamate, 50 mM Tris buffered with acetic acid to pH 8.2. S30B buffer comprises 14 mM Mg-glutamate, 150 mM K-glutamate buffered to pH 8.2 with Tris. Cell lysis was achieved by bead-beating for two intervals of 30 seconds using 0.1 mm glass beads and a Mini-Bead Beater (Biospec, Bartlesville, Okla.).

Expression plasmids and recombinant gene design and construction. The expression plasmid pBEST-OR2-OR1-Pr-UTR1-deGFP-T500 used in this study was described previously25. The plasmid was used for cell-free protein synthesis based on the activities of endogenous core RNA polymerase and sigma factor 70. The plasmids pBEST-OR2-OR1-Pr-UTR1-ADHhistag-T500 and pBEST-OR2-OR1-Pr-UTR1-CueOhistag-T500 were sub-cloned using common restriction-ligation methods. All plasmids were grown in E. coli DH5α cells and harvested using a Qiaprep spin miniprep kit (Qiagen, Hilden, Germany). To mutate various codons in various genes to amber nonsense codons, a KAPA HiFi PCR Kit was employed (Kapa Biosynthesis, Wilmington, Mass.) with a thermo-cycler (Bioer Technologies,). The resulting mutated PCR product was then heat shock transformed into competent E. coli DH5α cells (New England Biolabs) and plated onto selective plates to isolate transformed colonies. Suspected transformed colonies were sequentially incubated overnight and their plasmids were harvested and sequenced.

Cell-free protein synthesis. Cell-free reactions were carried out in volumes of 10 μL at 29° C. The 3-PGA reaction buffer is composed of 50 mM Hepes, pH 8, 1.5 mM ATP and GTP, 0.9 mM CTP and UTP, 0.2 mg/mL tRNA, 0.26 mM coenzyme A, 0.33 mM NAD, 0.75 mM cAMP, 0.068 mM folinic acid, 1 mM spermidine, 30 mM 3-phosphoglyceric acid, 1.5 mM each of 20 amino acids, 1 mM DTT, 2% PEG-8000. A typical cell-free reaction with this system contained 33% (by volume) E. coli extract, corresponding to a protein concentration of 10-15 mg/mL, before target protein synthesis. The other 66% of the reaction volume are composed of the plasmids, reaction buffer containing nutrients and the UAA. The concentrations of all reagents in the reaction buffer were fixed except for magnesium glutamate and potassium glutamate, containing two essential ions for CFPS and molecular interactions involved in transcription and translation. The cell-free expression system was prepared so as to adjust the concentrations of these two ions for a given strain extract. Optimization of these ions was achieved by performing deGFP CFPS in the presence of 1-6 mM Mg-Glutamate while fixing the K-glutamate concentration at a mean concentration of 80 mM and then sequentially optimizing the K-glutamate concentration between 20-140 mM. The genetically expanded cell free protein synthesis system was thus created by combining the cell free lysate, template DNA and the non-natural amino acids, as appropriate. Additional factors were added, such as additional natural amino acids, co-factors, nucleotides and energy solution and crowding agents, and reaction enhancement buffer. Once the system was prepared, template DNA was brought into proximity with the orthogonal pair, and the orthogonal pair suppressed/recognized it and added the UAA at the desired location via ribosomal translation.

When expressing deGFP, the reaction components were added to A Nunc 384 (120 μL)-well plates (Thermo Fisher Scientific) in order to sample deGFP expression as reflected by fluorescence intensity periodically (every 30 min). Reactions were incubated for no less than 10 h and fluorescence kinetics were measured. When expressing ADH or CueO, the reaction components were added to either 200 μL PCR tubes or 384-well plates and incubated in the thermo-cycler for 10 h. We found that CFPS reactions in well plates are very convenient for sequential down-stream processing and assessing activity.

For LC-MS validation of incorporation of PrK, nickel affinity chromatography purification of 6×his-tagged deGFP was performed. 500 μL of CFPS reaction mixture was incubated overnight at 29° C. to produce either deGFP Y35X (N-terminal 6×his tag) or WT deGFP (N-terminal 6×his tag). The reaction mixture was then diluted with 3 volumes of PB buffer (50 mM PB pH 8, 0.3 mM NaCl and 10 mM imidazole) and added to a nickel-bead column (Novagen, Madison, Wis.). Wash (50 mM imidazole) and elution (250 mM imidazole) steps were conducted according to the manufacturer's instructions. The protein-containing eluted fraction was concentrated using a Vivaspin 10 kDa cutoff concentrator (Sartorius, Göttingen, Germany) The resulting concentrated fraction was analyzed by LC-MS (Finnigan Surveyor Autosample Plus/LCQ Fleet, Thermo Scientific, Waltham, Mass.).

For the click chemistry downstream reaction, size exclusion-based purification of untagged proteins was performed. 120 μL of CFPS reaction mixture was incubated overnight at 29° to produce deGFP Y35X incorporating PrK. The reaction mixture was then diluted (×10) by DDW and subjected to size exclusion chromatography using an AKTA apparatus (GE Healthcare, Tel-Aviv, Israel) and the relevant 8 mL fraction was collected. The relevant fractions were determined prior to the purification of the reaction mixture by using commercially purified EGFP (MBL International, Woburn, Mass.). The fraction was concentrated using a Vivaspin 10 kDa cutoff concentrator (Sartorius, Göttingen, Germany) The resulting concentrated fraction was used for a “click” reaction.

“Click” reaction. The deGFP containing Propargyl-lysine was labeled using the Cu(I) catalyzed azide-alkyne cycloaddition reaction (CuAAC). Protein sample was resuspended in 0.1M PB pH=7.5. Tetramethylrhodamine-Azide (TAMRA-Az) (Sigma) was added to a concentration of 100 μM. THPTA, Sodium ascorbate and CuCl₂ were added to final concentrations of 400 μM, 2.5 mM and 200 ?uM, respectively. The reaction mixture was incubated at room temperature for from 3-12 hours. 20 μL sample from the mixture was diluted with 4×SDS sample buffer and kept for 10 min at 70° C., after which it was loaded and run on a 12% SDS-PAGE gel. Labeled proteins were visualized in-gel using ImageQuant LAS 4000 imager (Fujifilm, Tokyo, Japan), in fluorescence mode.

Alcohol Dehydrogenase activity assay. In a NUNC 384 well plate, 8.5 ul (42.5% volume) of CFPS reaction mixture was mixed with 7.5 ul (37.5% volume) 10 mg/ml NAD (Sigma Aldrich Co.;) TRIS solution (pH 8). Finally, 4 ul (20% volume) of the substrate EtOH (Absolute) was added and immediately inserted to the Platereader. The reaction was periodically shaken and 340 nm absorption was measured every 60 seconds for 20 minutes. The results represent the reduction of the absorption at t=0 and the peak absorption value measured

E. coli copper efflux oxidase activity assay. In a NUNC 384 well plate, 10 ul (50% volume) of CFPS reaction mixture was mixed with 10 ul of (50% volume) Sigma fast OPD (Sigma Aldrich Co.) TRIS solution (pH 8) and immediately inserted to the Platereader. The reaction was periodically shaken and 436 nm absorption was measured every 60 seconds for 20 minutes. The results represent the reduction of the absorption at t=0 and the peak absorption value measured.

The genes and plasmid sequences used in this study are as follows:

pBEST Plasmid containing the deGFP gene:
[SEQ ID NO: 1]
AATAATTTTGTTTAACTTTAAGAAGGAGATATACCATGGAGCTTTTCACT
GGCGTTGTTCCCATCCTGGTCGAGCTGGACGGCGACGTAAACGGCCACAA
GTTCAGCGTGTCCGGCGAGGGCGAGGGCGATGCCACCTACGGCAAGCTGA
CCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGA
CCACATGAAGCAGCACGACTTCTTCAAGTCCGCCATGCCCGAAGGCTACG
TCCAGGAGCGCACCATCTTCTTCAAGGACGACGGCAACTACAAGACCCGC
GCCGAGGTGAAGTTCGAGGGCGACACCCTGGTGAACCGCATCGAGCTGAA
GGGCATCGACTTCAAGGAGGACGGCAACATCCTGGGGCACAAGCTGGAGT
ACAACTACAACAGCCACAACGTCTATATCATGGCCGACAAGCAGAAGAAC
GGCATCAAGGTGAACTTCAAGATCCGCCACAACATCGAGGACGGCAGCGT
GCAGCTCGCCGACCACTACCAGCAGAACACCCCCATCGGCGACGGCCCCG
TGCTGCTGCCCGACAACCACTACCTGAGCACCCAGTCCGCCCTGAGCAAA
GACCCCAACGAGAAGCGCGATCACATGGTCCTGCTGGAGTTCGTGACCGC
CGCCGGGATCTAACTCGAGCAAAGCCCGCCGAAAGGCGGGCTTTTCTGTG
TCGACCGATGCCCTTGAGAGCCTTCAACCCAGTCAGCTCCTTCCGGTGGG
CGCGGGGCATGACTATCGTCGCCGCACTTATGACTGTCTTCTTTATCATG
CAACTCGTAGGACAGGTGCCGGCAGCGCTCTTCCGCTTCCTCGCTCACTG
ACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAAT
CGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGC
CGCTTACCGGATACCTGTCCGCCTTTCTCCCTTCGGGAAGCGTGGCGCTT
TCTCAATGCTCACGCTGTAGGTATCTCAGTTCGGTGTAGGTCGTTCGCTC
CAAGCTGGGCTGTGTGCACGAACCCCCCGTTCAGCCCGACCGCTGCGCCT
TATCCGGTAACTATCGTCTTGAGTCCAACCCGGTAAGACACGACTTATCG
CCACTGGCAGCAGCCACTGGTAACAGGATTAGCAGAGCGAGGTATGTAGG
CGGTGCTACAGAGTTCTTGAAGTGGTGGCCTAACTACGGCTACACTAGAA
GGACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAAC
TCACGTTAAGGGATTTTGGTCATGAGATTATCAAAAAGGATCTTCACCTA
GATCCTTTTAAATTAAAAATGAAGTTTTAAATCAATCTAAAGTATATATG
AGTAAACTTGGTCTGACAGTTACCAATGCTTAATCAGTGAGGCACCTATC
TCAGCGATCTGTCTATTTCGTTCATCCATAGTTGCCTGACTCCCCGTCGT
GTAGATAACTACGATACGGGAGGGCTTACCATCTGGCCCCAGTGCTGCAA
TGATACCGCGAGACCCACGCTCACCGGCTCCAGATTTATCAGCAATAAAC
CAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGC
CAGTTAATAGTTTGCGCAACGTTGTTGCCATTGCTACAGGCATCGTGGTG
TCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCT
TCGGTCCTCCGATCGTTGTCAGAAGTAAGTTGGCCGCAGTGTTATCACTC
ATGGTTATGGCAGCACTGCATAATTCTCTTACTGTCATGCCATCCGTAAG
ATGCTTTTCTGTGACTGGTGAGTACTCAACCAAGTCATTCTGAGAATAGT
GTATGCGGCGACCGAGTTGCTCTTGCCCGGCGTCAATACGGGATAATACC
GCGCCACATAGCAGAACTTTAAAAGTGCTCATCATTGGAAAACGTTCTTC
GGGGCGAAAACTCTCAAGGATCTTACCGCTGTTGAGATCCAGTTCGATGT
AACCCACTCGTGCACCCAACTGATCTTCAGCATCTTTTACTTTCACCAGC
GTTTCTGGGTGAGCAAAAACAGGAAGGCAAAATGCCGCAAAAAAGGGAAT
AAGGGCGACACGGAAATGTTGAATACTCATACTCTTCCTTTTTCAATATT
ATTGAAGCATTTATCAGGGTTATTGTCTCATGAGCGGATACATATTTGAA
TGTATTTAGAAAAATAAACAAATAGGGGTTCCGCGCACATTTCCCCGAAA
AGTGCCACCTGACGTCTAAGAAACCATTATTATCATGACATTAACCTATA
AAAATAGGCGTATCACGAGGCCCTTTCGTCTTCAAGAATTCTGGCGAATC
CTCTGACCAGCCAGAAAACGACCTTTCTGTGGTGAAACCGGATGCTGCAA
TTCAGAGCGGCAGCAAGTGGGGGACAGCAGAAGACCTGACCGCCGCAGAG
TGGATGTTTGACATGGTGAAGACTATCGCACCATCAGCCAGAAAACCGAA
TTTTGCTGGGTGGGCTAACGATATCCGCCTGATGCGTGAACGTGACGGAC
GTAACCACCGCGACATGTGTGTGCTGTTCCGCTGGGCATGCTGAGCTAAC
ACCGTGCGTGTTGACAATTTTACCTCTGGCGGTGATAATGGTTGCAGCTA
GC
deGFP:
[SEQ ID NO: 2]
ATGGAGCTTTTCACTGGCGTTGTTCCCATCCTGGTCGAGCTGGACGGCGA
CGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCC
GTGCCCTGGCCCACCCTCGTGACCACCCTGACCTACGGCGTGCAGTGCTT
CAGCCGCTACCCCGACCACATGAAGCAGCACGACTTCTTCAAGTCCGCCA
TGCCCGAAGGCTACGTCCAGGAGCGCACCATCTTCTTCAAGGACGACGGC
AACTACAAGACCCGCGCCGAGGTGAAGTTCGAGGGCGACACCCTGGTGAA
CCGCATCGAGCTGAAGGGCATCGACTTCAAGGAGGACGGCAACATCCTGG
GGCACAAGCTGGAGTACAACTACAACAGCCACAACGTCTATATCATGGCC
GACAAGCAGAAGAACGGCATCAAGGTGAACTTCAAGATCCGCCACAACAT
CGAGGACGGCAGCGTGCAGCTCGCCGACCACTACCAGCAGAACACCCCCA
TCGGCGACGGCCCCGTGCTGCTGCCCGACAACCACTACCTGAGCACCCAG
TCCGCCCTGAGCAAAGACCCCAACGAGAAGCGCGATCACATGGTCCTGCT
GGAGTTCGTGACCGCCGCCGGGATCTAA
pEVOL Pyl-OTS plasmid:
[SEQ ID NO: 3]
TCCTGAAAATCTCGATAACTCAAAAAATACGCCCGGTAGTGATCTTATTT
CATTATGGTGAAAGTTGGAACCTCTTACGTGCCGATCAACGTCTCATTTT
CGCCAAAAGTTGGCCCAGGGCTTCCCGGTATCAACAGGGACACCAGGATT
TATTTATTCTGCGAAGTGATCTTCCGTCACAGGTATTTATTCGGCGCAAA
GTGCGTCGGGTGATGCTGCCAACTTACTGATTTAGTGTATGATGGTGTTT
TTGAGGTGCTCCAGTGGCTTCTGTTTCTATCAGCTGTCCCTCCTGTTCAG
CTACTGACGGGGTGGTGCGTAACGGCAAAAGCACCGCCGGACATCAGCGC
TAGCGGAGTGTATACTGGCTTACTATGTTGGCACTGATGAGGGTGTCAGT
GAAGTGCTTCATGTGGCAGGAGAAAAAAGGCTGCACCGGTGCGTCAGCAG
AATATGTGATACAGGATATATTCCGCTTCCTCGCTCACTGACTCGCTACG
CTCGGTCGTTCGACTGCGGCGAGCGGAAATGGCTTACGAACGGGGCGGAG
ATTTCCTGGAAGATGCCAGGAAGATACTTAACAGGGAAGTGAGAGGGCCG
CGGCAAAGCCGTTTTTCCATAGGCTCCGCCCCCCTGACAAGCATCACGAA
ATCTGACGCTCAAATCAGTGGTGGCGAAACCCGACAGGACTATAAAGATA
CCAGGCGTTTCCCCCTGGCGGCTCCCTCGTGCGCTCTCCTGTTCCTGCCT
TTCGGTTTACCGGTGTCATTCCGCTGTTATGGCCGCGTTTGTCTCATTCC
ACGCCTGACACTCAGTTCCGGGTAGGCAGTTCGCTCCAAGCTGGACTGTA
TGCACGAACCCCCCGTTCAGTCCGACCGCTGCGCCTTATCCGGTAACTAT
CGTCTTGAGTCCAACCCGGAAAGACATGCAAAAGCACCACTGGCAGCAGC
CACTGGTAATTGATTTAGAGGAGTTAGTCTTGAAGTCATGCGCCGGTTAA
GGCTAAACTGAAAGGACAAGTTTTGGTGACTGCGCTCCTCCAAGCCAGTT
ACCTCGGTTCAAAGAGTTGGTAGCTCAGAGAACCTTCGAAAAACCGCCCT
GCAAGGCGGTTTTTTCGTTTTCAGAGCAAGAGATTACGCGCAGACCAAAA
CGATCTCAAGAAGATCATCTTATTAATCAGATAAAATATTTCTAGATTTC
AGTGCAATTTATCTCTTCAAATGTAGCACCTGAAGTCAGCCCCATACGAT
ATAAGTTGTAATTCTCATGTTTGACAGCTTATCATCGATAAGCTTGGTAC
CCAATTATGACAACTTGACGGCTACATCATTCACTTTTTCTTCACAACCG
GCACGGAACTCGCTCGGGCTGGCCCCGGTGCATTTTTTAAATACCCGCGA
GAAATAGAGTTGATCGTCAAAACCAACATTGCGACCGACGGTGGCGATAG
GCATCCGGGTGGTGCTCAAAAGCAGCTTCGCCTGGCTGATACGTTGGTCC
TCGCGCCAGCTTAAGACGCTAATCCCTAACTGCTGGCGGAAAAGATGTGA
CAGACGCGACGGCGACAAGCAAACATGCTGTGCGACGCTGGCGATATCAA
AATTGCTGTCTGCCAGGTGATCGCTGATGTACTGACAAGCCTCGCGTACC
CGATTATCCATCGGTGGATGGAGCGACTCGTTAATCGCTTCCATGCGCCG
CAGTAACAATTGCTCAAGCAGATTTATCGCCAGCAGCTCCGAATAGCGCC
CTTCCCCTTGCCCGGCGTTAATGATTTGCCCAAACAGGTCGCTGAAATGC
GGCTGGTGCGCTTCATCCGGGCGAAAGAACCCCGTATTGGCAAATATTGA
CGGCCAGTTAAGCCATTCATGCCAGTAGGCGCGCGGACGAAAGTAAACCC
ACTGGTGATACCATTCGCGAGCCTCCGGATGACGACCGTAGTGATGAATC
TCTCCTGGCGGGAACAGCAAAATATCACTCGGTCGGCAAACAAATTCTCG
TCCCTGATTTTTCACCACCCCCTGACCGCGAATGGTGAGATTGAGAATAT
AACCTTTCATTCCCAGCGGTCGGTCGATAAAAAAATCGAGATAACCGTTG
GCCTCAATCGGCGTTAAACCCGCCACCAGATGGGCATTAAACGAGTATCC
CGGCAGCAGGGGATCATTTTGCGCTTCAGCCATACTTTTCATACTCCCGC
CATTCAGAGAAGAAACCAATTGTCCATATTGCATCAGACATTGCCGTCAC
TGCGTCTTTTACTGGCTCTTCTCGCTAACCAAACCGGTAACCCCGCTTAT
TAAAAGCATTCTGTAACAAAGCGGGACCAAAGCCATGACAAAAACGCGTA
ACAAAAGTGTCTATAATCACGGCAGAAAAGTCCACATTGATTATTTGCAC
GGCGTCACACTTTGCTATGCCATAGCATTTTTATCCATAAGATTAGCGGA
TCCTACCTGACGCTTTTTATCGCAACTCTCTACTGTTTCTCCATACCCGT
TTTTTTGGGCTAACAGGAGGAATTAGATCTATGGATAAAAAACCACTAAA
CACTCTGATCTCTGCTACTGGTCTGTGGATGAGTCGTACCGGAACCATTC
ATAAAATCAAACACCACGAGGTTAGCCGTTCGAAAATCTATATTGAGATG
GCGTGTGGCGATCATCTGGTTGTGAACAATAGCCGCTCTTCTCGTACAGC
ACGTGCACTGCGTCACCACAAATATCGTAAAACCTGTAAACGTTGCCGTG
TGTCCGATGAGGATCTGAACAAATTCCTGACAAAAGCCAATGAGGACCAA
ACAAGCGTGAAAGTGAAAGTCGTTAGCGCTCCTACCCGTACTAAAAAAGC
AATGCCGAAATCCGTTGCTCGTGCCCCTAAACCACTGGAAAACACTGAAG
CAGCACAGGCACAGCCGTCTGGAAGCAAATTCTCTCCGGCCATTCCTGTT
TCTACCCAGGAGTCCGTTTCTGTTCCAGCAAGTGTGAGCACCAGCATTAG
CAGTATTAGCACCGGTGCCACCGCTAGCGCCCTGGTTAAAGGCAATACCA
ATCCGATTACAAGCATGTCTGCCCCGGTTCAAGCATCAGCTCCAGCACTG
ACAAAATCCCAAACCGATCGTCTGGAGGTTCTGCTGAATCCGAAAGACGA
AATCAGCCTGAATTCCGGCAAACCGTTTCGTGAACTGGAGAGCGAACTGC
TGTCACGTCGTAAAAAAGACCTGCAACAAATCTATGCCGAAGAACGTGAG
AACTATCTGGGGAAACTGGAACGTGAAATCACCCGCTTTTTCGTGGATCG
TGGCTTTCTGGAGATCAAATCCCCGATTCTGATTCCTCTGGAGTATATCG
AGCGTATGGGCATCGACAATGATACCGAACTGAGCAAACAAATTTTCCGT
GTGGATAAAAACTTCTGTCTGCGCCCTATGCTGGCACCAAATCTGTATAA
CTATCTGCGCAAACTGGACCGTGCCCTGCCTGATCCTATCAAAATCTTCG
AGATCGGCCCGTGTTATCGTAAAGAGTCCGACGGTAAAGAACATCTGGAG
GAGTTTACCATGCTGAACTTTTGCCAAATGGGTTCAGGTTGTACTCGTGA
GAACCTGGAAAGCATCATCACCGATTTTCTGAACCACCTGGGCATTGACT
TCAAAATTGTGGGCGACAGCTGTATGGTGTATGGCGACACCCTGGATGTC
ATGCACGGCGACCTGGAACTGTCTAGTGCCGTTGTTGGACCAATTCCGCT
GGACCGTGAGTGGGGTATCGACAAACCGTGGATCGGAGCAGGATTCGGTC
TGGAACGCCTGCTGAAAGTGAAACACGACTTCAAAAACATCAAACGTGCC
GCCCGTTCTGAATCGTATTATAACGGGATTTCTACCAACCTGTAAGTCGA
CCATCATCATCATCATCATTGAGTTTAAACGGTCTCCAGCTTGGCTGTTT
TGGCGGATGAGAGAAGATTTTCAGCCTGATACAGATTAAATCAGAACGCA
GAAGCGGTCTGATAAAACAGAATTTGCCTGGCGGCAGTAGCGCGGTGGTC
CCACCTGACCCCATGCCGAACTCAGAAGTGAAACGCCGTAGCGCCGATGG
TAGTGTGGGGTCTCCCCATGCGAGAGTAGGGAACTGCCAGGCATCAAATA
AAACGAAAGGCTCAGTCGAAAGACTGGGCCTTGTTTGTGAGCTCCCGGTC
ATCAATCATCCCCATAATCCTTGTTAGATTATCAATTTTAAAAAACTAAC
AGTTGTCAGCCTGTCCCGCTTTAATATCATACGCCGTTATACGTTGTTTA
CGCTTTGAGGAATCCCATATGGATAAAAAACCACTAAACACTCTGATCTC
TGCTACTGGTCTGTGGATGAGTCGTACCGGAACCATTCATAAAATCAAAC
ACCACGAGGTTAGCCGTTCGAAAATCTATATTGAGATGGCGTGTGGCGAT
CATCTGGTTGTGAACAATAGCCGCTCTTCTCGTACAGCACGTGCACTGCG
TCACCACAAATATCGTAAAACCTGTAAACGTTGCCGTGTGTCCGATGAGG
ATCTGAACAAATTCCTGACAAAAGCCAATGAGGACCAAACAAGCGTGAAA
GTGAAAGTCGTTAGCGCTCCTACCCGTACTAAAAAAGCAATGCCGAAATC
CGTTGCTCGTGCCCCTAAACCACTGGAAAACACTGAAGCAGCACAGGCAC
AGCCGTCTGGAAGCAAATTCTCTCCGGCCATTCCTGTTTCTACCCAGGAG
TCCGTTTCTGTTCCAGCAAGTGTGAGCACCAGCATTAGCAGTATTAGCAC
CGGTGCCACCGCTAGCGCCCTGGTTAAAGGCAATACCAATCCGATTACAA
GCATGTCTGCCCCGGTTCAAGCATCAGCTCCAGCACTGACAAAATCCCAA
ACCGATCGTCTGGAGGTTCTGCTGAATCCGAAAGACGAAATCAGCCTGAA
TTCCGGCAAACCGTTTCGTGAACTGGAGAGCGAACTGCTGTCACGTCGTA
AAAAAGACCTGCAACAAATCTATGCCGAAGAACGTGAGAACTATCTGGGG
AAACTGGAACGTGAAATCACCCGCTTTTTCGTGGATCGTGGCTTTCTGGA
GATCAAATCCCCGATTCTGATTCCTCTGGAGTATATCGAGCGTATGGGCA
TCGACAATGATACCGAACTGAGCAAACAAATTTTCCGTGTGGATAAAAAC
TTCTGTCTGCGCCCTATGCTGGCACCAAATCTGTATAACTATCTGCGCAA
ACTGGACCGTGCCCTGCCTGATCCTATCAAAATCTTCGAGATCGGCCCGT
GTTATCGTAAAGAGTCCGACGGTAAAGAACATCTGGAGGAGTTTACCATG
CTGAACTTTTGCCAAATGGGTTCAGGTTGTACTCGTGAGAACCTGGAAAG
CATCATCACCGATTTTCTGAACCACCTGGGCATTGACTTCAAAATTGTGG
GCGACAGCTGTATGGTGTATGGCGACACCCTGGATGTCATGCACGGCGAC
CTGGAACTGTCTAGTGCCGTTGTTGGACCAATTCCGCTGGACCGTGAGTG
GGGTATCGACAAACCGTGGATCGGAGCAGGATTCGGTCTGGAACGCCTGC
TGAAAGTGAAACACGACTTCAAAAACATCAAACGTGCCGCCCGTTCTGAA
TCGTATTATAACGGGATTTCTACCAACCTGTAACTGCAGTTTCAAACGCT
AAATTGCCTGATGCGCTACGCTTATCAGGCCTACATGATCTCTGCAATAT
ATTGAGTTTGCGTGCTTTTGTAGGCCGGATAAGGCGTTCACGCCGCATCC
GGCAAGAAACAGCAAACAATCCAAAACGCCGCGTTCAGCGGCGTTTTTTC
TGCTTTTCTTCGCGAATTAATTCCGCTTCGCAACATGTGAGCACCGGTTT
ATTGACTACCGGAAGCAGTGTGACCGTGTGCTTCTCAAATGCCTGAGGCC
AGTTTGCTCAGGCTCTCCCCGTGGAGGTAATAATTGACGATATGATCAGT
GCACGGCTAACTAAGCGGCCTGCTGACTTTCTCGCCGATCAAAAGGCATT
TTGCTATTAAGGGATTGACGAGGGCGTATCTGCGCAGTAAGATGCGCCCC
GCATTTATGCATGGCGATATCTAATACGACTCACTATAGGAAACCTGATC
ATGTAGATCGAATGGACTCTAAATCCGTTCAGCCGGGTTAGATTCCCGGG
GTTTCCGCCAAATTCGAAAAGCCTGCTCAACGAGCAGGCTTTTTTGCATG
CTCGAGCAGCTCAGGGTCGAATTTGCTTTCGAATTTCTGCCATTCATCCG
CTTATTATCACTTATTCAGGCGTAGCACCAGGCGTTTAAGGGCACCAATA
ACTGCCTTAAAAAAATTACGCCCCGCCCTGCCACTCATCGCAGTACTGTT
GTAATTCATTAAGCATTCTGCCGACATGGAAGCCATCACAGACGGCATGA
TGAACCTGAATCGCCAGCGGCATCAGCACCTTGTCGCCTTGCGTATAATA
TTTGCCCATGGTGAAAACGGGGGCGAAGAAGTTGTCCATATTGGCCACGT
TTAAATCAAAACTGGTGAAACTCACCCAGGGATTGGCTGAGACGAAAAAC
ATATTCTCAATAAACCCTTTAGGGAAATAGGCCAGGTTTTCACCGTAACA
CGCCACATCTTGCGAATATATGTGTAGAAACTGCCGGAAATCGTCGTGGT
ATTCACTCCAGAGCGATGAAAACGTTTCAGTTTGCTCATGGAAAACGGTG
TAACAAGGGTGAACACTATCCCATATCACCAGCTCACCGTCTTTCATTGC
CATACGGAATTCCGGATGAGCATTCATCAGGCGGGCAAGAATGTGAATAA
AGGCCGGATAAAACTTGTGCTTATTTTTCTTTACGGTCTTTAAAAAGGCC
GTAATATCCAGCTGAACGGTCTGGTTATAGGTACATTGAGCAACTGACTG
AAATGCCTCAAAATGTTCTTTACGATGCCATTGGGATATATCAACGGTGG
TATATCCAGTGATTTTTTTCTCCATTTTAGCTTCCT
Methanosarcina mazei PylRS:
[SEQ ID NO: 4]
ATGGATAAAAAACCACTAAACACTCTGATCTCTGCTACTGGTCTGTGGAT
GAGTCGTACCGGAACCATTCATAAAATCAAACACCACGAGGTTAGCCGTT
CGAAAATCTATATTGAGATGGCGTGTGGCGATCATCTGGTTGTGAACAAT
AGCCGCTCTTCTCGTACAGCACGTGCACTGCGTCACCACAAATATCGTAA
AACCTGTAAACGTTGCCGTGTGTCCGATGAGGATCTGAACAAATTCCTGA
CAAAAGCCAATGAGGACCAAACAAGCGTGAAAGTGAAAGTCGTTAGCGCT
CCTACCCGTACTAAAAAAGCAATGCCGAAATCCGTTGCTCGTGCCCCTAA
ACCACTGGAAAACACTGAAGCAGCACAGGCACAGCCGTCTGGAAGCAAAT
TCTCTCCGGCCATTCCTGTTTCTACCCAGGAGTCCGTTTCTGTTCCAGCA
AGTGTGAGCACCAGCATTAGCAGTATTAGCACCGGTGCCACCGCTAGCGC
CCTGGTTAAAGGCAATACCAATCCGATTACAAGCATGTCTGCCCCGGTTC
AAGCATCAGCTCCAGCACTGACAAAATCCCAAACCGATCGTCTGGAGGTT
CTGCTGAATCCGAAAGACGAAATCAGCCTGAATTCCGGCAAACCGTTTCG
TGAACTGGAGAGCGAACTGCTGTCACGTCGTAAAAAAGACCTGCAACAAA
TCTATGCCGAAGAACGTGAGAACTATCTGGGGAAACTGGAACGTGAAATC
ACCCGCTTTTTCGTGGATCGTGGCTTTCTGGAGATCAAATCCCCGATTCT
GATTCCTCTGGAGTATATCGAGCGTATGGGCATCGACAATGATACCGAAC
TGAGCAAACAAATTTTCCGTGTGGATAAAAACTTCTGTCTGCGCCCTATG
CTGGCACCAAATCTGTATAACTATCTGCGCAAACTGGACCGTGCCCTGCC
TGATCCTATCAAAATCTTCGAGATCGGCCCGTGTTATCGTAAAGAGTCCG
ACGGTAAAGAACATCTGGAGGAGTTTACCATGCTGAACTTTTGCCAAATG
GGTTCAGGTTGTACTCGTGAGAACCTGGAAAGCATCATCACCGATTTTCT
GAACCACCTGGGCATTGACTTCAAAATTGTGGGCGACAGCTGTATGGTGT
ATGGCGACACCCTGGATGTCATGCACGGCGACCTGGAACTGTCTAGTGCC
GTTGTTGGACCAATTCCGCTGGACCGTGAGTGGGGTATCGACAAACCGTG
GATCGGAGCAGGATTCGGTCTGGAACGCCTGCTGAAAGTGAAACACGACT
TCAAAAACATCAAACGTGCCGCCCGTTCTGAATCGTATTATAACGGGATT
TCTACCAACCTGTAA
Methanosarcina mazei Pyl-tRNA_{cua}^{PYl}:
[SEQ ID NO: 5]
GGAAACCTGATCATGTAGATCGAATGGACTCTAAATCCGTTCAGCCGGGT
TAGATTCCCGGGGTTTCCGCCA
Zymomonas mobilis ADHII:
[SEQ ID NO: 6]
ATGGCTTCTTCAACTTTTTATATTCCTTTCGTCAACGAAATGGGCGAAGG
TTCGCTTGAAAAAGCAATCAAGGATCTTAACGGCAGCGGCTTTAAAAATG
CCCTGATCGTTTCTGATGCTTTCATGAACAAATCCGGTGTTGTGAAGCAG
GTTGCTGACCTGTTGAAAACACAGGGTATTAATTCTGCTGTTTATGATGG
CGTTATGCCGAACCCGACTGTTACCGCAGTTCTGGAAGGCCTTAAGATCC
TGAAGGATAACAATTCAGACTTCGTCATCTCCCTCGGTGGTGGTTCTCCC
CATGACTGCGCCAAAGCCATCGCTCTGGTCGCAACCAATGGTGGTGAAGT
CAAAGACTACGAAGGTATCGACAAATCTAAGAAACCTGCCCTGCCTTTGA
TGTCAATCAACACGACGGCTGGTACGGCTTCTGAAATGACGCGTTTCTGC
ATCATCACTGATGAAGTCCGTCACGTTAAGATGGCCATTGTTGACCGTCA
CGTTACCCCGATGGTTTCCGTCAACGATCCTCTGTTGATGGTTGGTATGC
CAAAAGGCCTGACCGCCGCCACCGGTATGGATGCTCTGACCCACGCATTT
GAAGCTTATTCTTCAACGGCAGCTACTCCGATCACCGATGCTTGCGCTTT
GAAAGCAGCTTCCATGATCGCTAAGAATCTGAAGACCGCTTGCGACAACG
GTAAGGATATGCCAGCTCGTGAAGCTATGGCTTATGCCCAATTCCTCGCT
GGTATGGCCTTCAACAACGCTTCGCTTGGTTATGTCCATGCTATGGCTCA
CCAGTTGGGCGGTTACTACAACCTGCCGCATGGTGTCTGCAACGCTGTTC
TGCTTCCGCATGTTCTGGCTTATAACGCCTCTGTCGTTGCTGGTCGTCTG
AAAGACGTTGGTGTTGCTATGGGTCTCGATATCGCCAATCTCGGCGATAA
AGAAGGCGCAGAAGCCACCATTCAGGCTGTTCGCGATCTGGCTGCTTCCA
TTGGTATTCCAGCAAATCTGACCGAGCTGGGTGCTAAGAAAGAAGATGTG
CCGCTTCTTGCTGACCACGCTCTGAAAGATGCTTGTGCTCTGACCAACCC
GCGTCAGGGTGATCAGAAAGAAGTTGAAGAACTCTTCCTGAGCGCTTTCT
AA
E.coli-CueO:\
[SEQ ID NO: 7]
ATGGCAGAACGCCCAACGTTACCGATCCCTGATTTGCTCACGACCGATGC
CCGTAATCGCATTCAGTTAACTATTGGCGCAGGCCAGTCCACCTTTGGCG
GGAAAACTGCAACTACCTGGGGCTATAACGGCAATCTGCTGGGGCCGGCG
GTGAAATTACAGCGCGGCAAAGCGGTAACGGTTGATATCTACAACCAACT
GACGGAAGAGACAACGTTGCACTGGCACGGGCTGGAAGTACCGGGTGAAG
TCGACGGCGGCCCGCAGGGAATTATTCCGCCAGGTGGCAAGCGCTCGGTG
ACGTTGAACGTTGATCAACCTGCCGCTACCTGCTGGTTCCATCCGCATCA
GCACGGCAAAACCGGGCGACAGGTGGCGATGGGGCTGGCTGGGCTGGTGG
TGATTGAAGATGACGAGATCCTGAAATTAATGCTGCCAAAACAGTGGGGT
ATCGATGATGTTCCGGTGATCGTTCAGGATAAGAAATTTAGCGCCGACGG
GCAGATTGATTATCAACTGGATGTGATGACCGCCGCCGTGGGCTGGTTTG
GCGATACGTTGCTGACCAACGGTGCAATCTACCCGCAACACGCTGCCCCG
CGTGGTTGGCTGCGCCTGCGTTTGCTCAATGGCTGTAATGCCCGTTCGCT
CAATTTCGCCACCAGCGACAATCGCCCGCTGTATGTGATTGCCAGCGACG
GTGGTCTGCTACCTGAACCAGTGAAGGTGAGCGAACTGCCGGTGCTGATG
GGCGAGCGTTTTGAAGTGCTGGTGGAGGTTAACGATAACAAACCCTTTGA
CCTGGTGACGCTGCCGGTCAGCCAGATGGGGATGGCGATTGCGCCGTTTG
ATAAGCCTCATCCGGTAATGCGGATTCAGCCGATTGCTATTAGTGCCTCC
GGTGCTTTGCCAGACACATTAAGTAGCCTGCCTGCGTTACCTTCGCTGGA
AGGGCTGACGGTACGCAAGCTGCAACTCTCTATGGACCCGATGCTCGATA
TGATGGGGATGCAGATGCTAATGGAGAAATATGGCGATCAGGCGATGGCC
GGGATGGATCACAGCCAGATGATGGGCCATATGGGGCACGGCAATATGAA
TCATATGAACCACGGCGGGAAGTTCGATTTCCACCATGCCAACAAAATCA
ACGGTCAGGCGTTTGATATGAACAAGCCGATGTTTGCGGCGGCGAAAGGG
CAATACGAACGTTGGGTTATCTCTGGCGTGGGCGACATGATGCTGCATCC
GTTCCATATCCACGGCACGCAGTTCCGTATCTTGTCAGAAAATGGCAAAC
CGCCAGCGGCTCATCGCGCGGGCTGGAAAGATACCGTTAAGGTAGAAGGT
AATGTCAGCGAAGTGCTGGTGAAGTTTAATCACGATGCACCGAAAGAACA
TGCTTATATGGCGCACTGCCATCTGCTGGA.

### Example 1

### Effective Cell Free Protein Synthesis Incorporating a Non-Natural Amino Acid: Results from a Reporter System

The recoded E. coli strain (GRO):C321.ΔprfA was utilized as it promotes the replacement of the message encoded by the amber nonsense codon, i.e. genomic TAG stop codons are replaced and the translation of a UAG triplet is translated as a sense codon, instead of as a nonsense codon.

The medium-low copy number pEVOL plasmid containing the OTS genes; mM-PylRS/mM-tRNA_{cua}^{pyl} was transformed to the GRO strain. This vector “pyl-OTS”, and several other OTS plasmids used in this study tested are specified hereinbelow in Table 1 and 2.

TABLE 1
A matrix describing the different transformations performed
in each experiment conducted in this study

| | pEVOL | pEVOL | pSUP | pKD | |
|---|---|---|---|---|---|
| | PylRS | PylRS-AF | pACF | SepRS | |
| Plasmid/ | (Pyl- | (PylAF- | (pACF- | (Sep- | No |
| Strain | OTS)^{a} | OTS)^{b} | OTS)^{c} | OTS)^{d} | OTS |
| C321.ΔprfA | + | + | + | + | + |
| C321. ΔprfA EXP | + | | | | |
| C321 (i.e. prfA+) | + | | | | |
| BL21(DE3) | + | | | | |
| DH5α | + | | | | |

^{a}Pyl-OTS: Mm-pyrrolysil synthetase/Mm-tRNA_{CUA}^{Pyl} (Blight et al., Nature 431: 333-335; Srinivasan et al., 2002 Science 296: 1459-1462)
^{b}PylAF-OTS: Mm-pyrrolysil synthetase/Mm-tRNA_{CUA}^{Pyl} (Herner et al., 2013 Org. Biomol. Chem. 11: 3297-3306).
^{c}pAcF-OTS: Mj-para-aceto-phenylalanyl synthetase/Mj-tRNA_{CUA}^{Opt} (Wang et al.,. 2001 Science 292: 498-500).
^{d}Sep-OTS: Mm-phospho-seryl synthetase/Mm-tRNA_{CUA}^{Sep} and Ef-sep (an orthogonal elongation factor) (Park et al., 2011 Science 333: 1151-1154).

TABLE 2
Strains used for CFPS extract preparation and plasmids used both as OTS
in the extract strain and as expression template for the CFPS Reactions.

| | | |
|---|---|---|
| Strains/ | | |
| Plasmids | Details, Use & Rationale of use | References |

Strains

| | | |
|---|---|---|
| C321.ΔRF1 | Genomically receded E. coli having all (321) | Lajoie, M., et. al. |
| | TAG nonsense codons replaced and release | Science 342, 357- |
| | factor 1 (RF1) knockout, making it ideal for | 360 (2013) |
| | Amber suppression (genetic code expansion). | (Addgene #48998) |
| | Herein, because of its attributes used as main | |
| | chassis. (Cm^{R}) | |
| C321.RF1+ | Same as above, but release factor 1 has not | Ibid, (Addgene |
| | been deleted. Used as a control for the effect | #48999) |
| | of RF1 on suppression efficiency. (Cm^{R}) | |
| DH5a | (F- endA1, glnV44, thi-1, recA1, relA1, gyrA96, | Phue J-N, et. al. |
| | deoR, nupG, φ80dlacZAM15, Δ(lacZYA- | Biotechnol. Bioeng. |
| | argF)U169, hsdR17(rK− mK+), E. coli strain that | 101: 831-836; |
| | transforms with high efficiency. Like many | Taylor, R. G., et. al. |
| | cloning strains, DH5 alpha has several features | 21, 1677-1678 |
| | that make it useful for recombinant DNA | (1993). |
| | methods.. | Phue, J.-N., et. al. |
| | | Biotechnol. Bioeng. |
| | | 101, 831-6 (2008) |
| | | (NEB product |
| | | #C2987H) |
| BL21(DE3) | (F_, ompT, hsdSB (rB_, mB), dcm, gal) E. coli | Phue, Ibid. |
| | strain that express proteins (Also under T7 | |
| | promoters) and replicates plasmid DNA with | |
| | high efficiency. | |

Plasmids

| | | |
|---|---|---|
| pEVOL | Orthogonal translation system (OTS) | Young, T. S., et. al. |
| MmPylRS/ | plasmid containing the MbPylRS gene under | J. Mol. Biol. 395, |
| MmPyltRNA | the regulation of araBAD promoter (induced | 361-374 (2010) |
| (i.e. the | by arabinose) and the MbPylT gene under the | |
| Pyl-OTS) | regulation of the proK promoter. (Cm^{R}) | |
| pEVOL | Same as above but the MbPylRS gene | Herner, A., et. al. |
| MmPylRS-AF/ | mutated to accept 1,3-benzothiazole | Org. Biomol. |
| MmPyltRNA | (bioorthogonal fluorescent dyes) derivatives | Chem. 11, 3297- |
| | (Cm^{R}) | 306 (2013) |
| pSUP pAcF | Orthogonal translation system (OTS) | Ryu, Y. & Schultz, |
| | plasmid containing the MjTyrRS (pACF) | P. G. Efficient |
| | gene under the regulation of glnS promoter | incorporation of |
| | and 6 copies of the MjTyrT genes under the | unnatural amino |
| | regulation of 2 different proK promoters. | acids into proteins |
| | (Cm^{R}) | in Escherichia coli. |
| | | 3, 263-266 (2006). |
| pKD SepRS, | Orthogonal translation system (OTS) used in | Park, H.-S. et al. |
| EFSep, 5x | phosphoprotein synthesis. Expresses the MjSep- | Science 333, 1151- |
| tRNASep | accepting tRNA (tRNASep), the M. Maripaludis | 4 (2011) (Addgene |
| (B40 OTS) | Sep-tRNA synthetase (SepRS) and an engineered | #52054) |
| | EF-Tu (EFSep) (Kan^{R}) | |
| pBEST- | Expression plasmid, deGFP expression is | Sun, Z. Z. et al.. J. |
| OR2-OR1- | regulated by the OR2-OR1 promoter | Vis. Exp. 1-15 |
| Pr-UTR1- | (bacteriophage Lambda promoter with one | (2013). |
| deGFP-T500 | mutation). The deGFP gene was mutated to | doi: 10.3791/50762 |
| | create the following variants: Y35X (i.e. | (Addgene #40019) |
| | Y35TAG mutation) (Amp^{R}) | |

In order to demonstrate successful incorporation of the non-natural Propargyl-Lysine (UAA) amino acid site specifically into a target protein, a destabilized eGFP variant that undergoes degradation (deGFP) was utilized, which was expressed under the control of the OR2-OR1-PR promoter. Thus the deGFP-encoding gene was sub-cloned into the pBEST-OR2-OR1-Pr-UTR1-deGFP-T500 plasmid (Addgene #40019) (Table 2) under the control of a mutated bacteriophage λ promoter (OR2-OR1-Pr), including mutagenesis of the Y35X codon position (where X denotes TAG).

Following preparation of the cell free protein synthesis system as described above utilizing the pEVOL derivative plasmids expressing the orthogonal translation system (OTS) in the E. coli strain C321.ΔPrfA, cell free protein synthesis was carried out and Western blot analysis was conducted probing the proteins produced using the genetically expanded CFPS system probing for GFP expression.

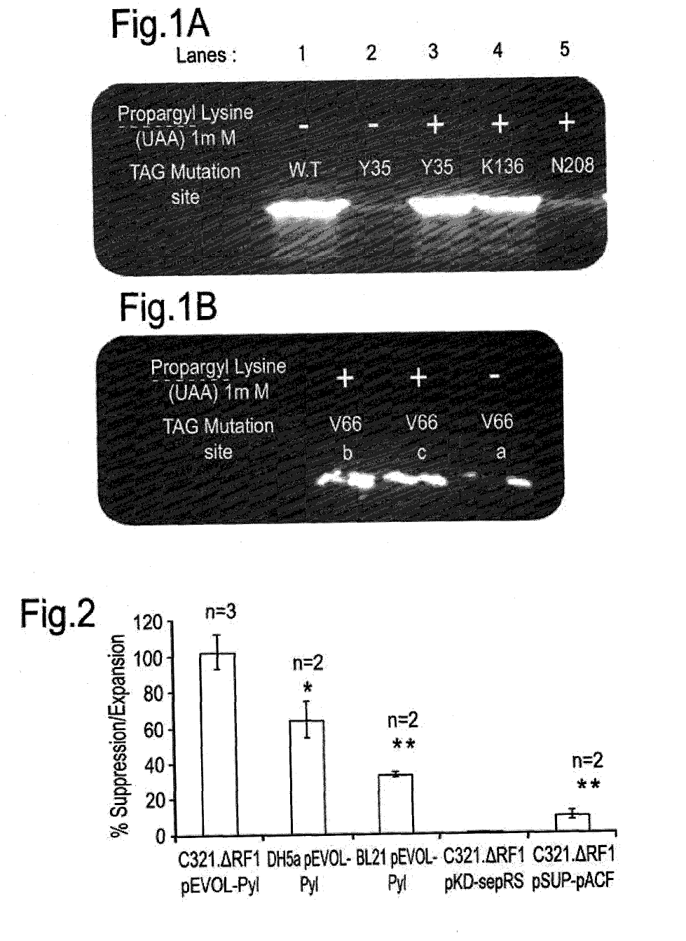

FIG. 1A shows the results of the Western Blot analysis. Lanes 1 and 2 contain WT and TAG mutation site Y35 samples, respectively, which were not provided with 1 mM Propargyl Lysine (UAA) as part of the cell free system.

Thus, only the WT deGFP is detected.

When 1 mM Propargyl Lysine (UAA) was provided as part of the cell free system in TAG mutation site Y35 and K136 samples, the deGFP was detected (lanes 3 and 4).

TAG mutation in the N208 site served was revealed as a non-permissive site—in other words, a site in the protein that when mutated to TAG results in an inability to be translated by the ribosome. FIG. 1B shows ADH expression at comparable levels in TAG mutation site V66 b and V66 c samples when 1 mM Propargyl Lysine (UAA) was provided as part of the cell free system, but not in V66 a samples.

FIG. 2 plots the comparison of suppression efficiencies between the different E. coli strains assessed.

As is evident from the figure, suppression efficiency gave the highest results when the E. coli strain used was C321.ΔPrfA. The suppression efficiency of the OTS is commonly calculated as:

[W.T protein]/[TAG containing protein]*100.

To understand and characterize the benefits gained by using the C321. ΔPrfA strain and to understand the effects of Release Factor 1 on the system various cell free extracts were produced, in order to compare their suppression efficiencies.

Toward this end, the pEVOL-Pyl OTS was compared with 3 E. coli strains [Table 1]; Dh5alpha, BL21(De3) and C321.Δ PrfA.

The comparison between RF1+ strains (Dh5alpha and BL21[De3]) and the RF1− strain provided unexpected findings in terms of the identified crucial components which enable system function.

Toward this end, crude extracts with varying parameters as described in Table 1 were prepared. By creating and experimenting with uninduced (Arabinose—the inducer was added during bacterial growth in all extract preparations except the former) C321.ΔPrfA pEVOL-pyl extract, the effects of induction of the pylRS facilitated in pEVOL OTS were tested. By creating and experimenting with the C321 (RF1+) strain, the effects of the RF1 amber suppressor on the behavior of the system and the suppression efficiency were evaluated. Attempts to create other C321.ΔPrfA OTS strains; i.e. pSUP-pACF OTS (Para-acetyl-Phenylalanine) and pKD-Sep (Phospho-Serine) OTS, were unsuccessful and therefore the compatibility of plasmids and other OTS were tested in this context.

From the results it is clear that the C321.ΔPrfA pEVOL-pyl system exhibits seamless suppression efficiency while the same OTS in other strains provides much lower results. The significant efficiency of this system was unexpected.

Moreover, when comparing the between the RF1+ and the RF1− C321 recoded strains the results thus obtained are that much more surprising.

Thus, the absence of the RF1 amber suppressor may play an important role in the advance in the system's suppression efficiency.

The reduced efficiency of the uninduced C321.ΔPrfA pEVOL-pyl strain could be explained by the lower cellular concentrations of the pyrrolysyl tRNA synthetase which sequentially reduces the rate of o-tRNA aminoacylation with the UAA and finally the overall yield of the genetically expanded CFPS [FIG. 1].

Taken together, the results indicate that using a TAG recoded organism with RF1 deletion, it is more appropriate to use of the same suppression efficiency equation to describe the “Expansion efficiency” between the original meaning of the TAG codon and the new meaning ascertained from the endogenous OTS.

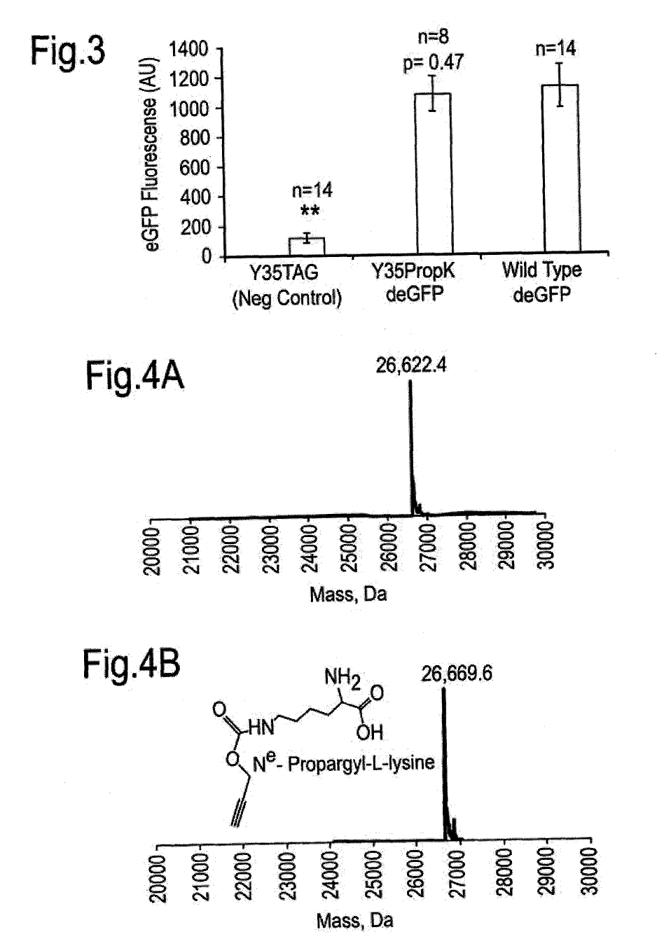

FIG. 3 further extends these results by plotting the relative fluorescence obtained when the cell free protein synthesis of the deGFP was assessed using the E. coli strain C321.ΔPrfA CFPS. When comparing the Y35PropK deGFP containing system versus the Y35TAG containing system, an expansion efficiency of almost 100% is obtained, approaching that of wild type deGFP in terms of fluorescence.

The results provide an indicator regarding system stability.

One key challenge in genetically expanded CFPS systems is the fact that by using exogenous components the overall stability of the system is reduced, serving as a major challenge in terms of the ability to reproduce the same quality and exact quantity of the exogenous OTS components (o-tRNA and o-aaRS) to be added to the CFPS reaction, and thereby limit industrial applicability. Moreover, storage time for the components, preserving activity of same is quite short (i.e. several weeks) resulting in frequent need to refresh stock [Table 3].

### Table 3. A Comparison Between Genetic Code System/Methodologies

TABLE 3
Comparison between genetic code system/methodologies

| | Endogenous Code | Exogenous code | |
|---|---|---|---|
| Item | expanding CFPS | expanding CFPS | In vivo |
| Time from PCR | Reaction: | tRNA synthesis: 2 days | Co-Transformation: |
| product to protein | Overnight | aaRS synthesis: 2 days | 1 day |
| expression | | Reaction: Overnight | Expression: 1 day |
| | | | Purification: 1 day |
| Expression vector | No | No | Yes |
| needed | | | |
| Amount of UAA | ~1 | ~1 μmol(UAA)/ | ~100 μmol(UAA)/ |
| needed | | mg(Protein) | mg(Protein) |
| Storage time | <1 year | o-tRNA: ~1.5 month | N/A |
| | | o-aaRS: ~2-4 weeks | |
| o-tRNA maturation | Complete | Either synthetic or cell | Complete |
| and nativity | | based but purified using | |
| | | organic solvents. | |
| aaRS folding and | Complete | Purification tags and | Complete |
| nativity | | processes needed | |
| Use of insoluble | Possible | Not reported | Possible |
| molecules (PylRS | | | |
| derivatives) | | | |
| Reaction preparation | ~30 minutes | ~1 hour | NA |
| time | | | |
| No. of different | 3 (DNA, | 5 (DNA, lysate, Buffer, tRNA | NA |
| component/processes | Lysate and | and aaRS). Last two items | |
| (Levels of | reaction | could go wrong both | |
| complexity) | buffer) | upstream and in downstream | |
| | | applications. | |
| Downstream | Instantly ready for immune assays, protein | | Needs to undergo |
| processes of product | assays, chemical reactions, calorimetric | | lysis before ready |
| | assays and purification. | | for downstream. |
| Tracking kinetics of | Using fluorescent tags or calorimetric assays | | No kinetic tracking. |
| protein expression | enable live tracking of expression kinetics | | |
| Reproducibility of | High | Medium | High |
| results | | | |
| Absolute Protein | >1 mg | >1 mg | <1 mg |
| Yields | | | |
| Relative Protein | ~1 mg/ml | ~1 mg/ml | 0.02 mg/ml |
| Yields | (reaction) | (reaction) | (culture) |
| Scale-up | Complex - But have 2 | Complex | Easy |
| | components less to | | |
| | independently | | |
| | produce and scale up | | |
| Commercialization | High | Low | N/A |
| potential | | | |

| | | |
|---|---|---|
| Simultaneous | Limitless, can easily create arrays. | Limited - |
| reactions with | | transformation, |
| different DNA | | growth and sorting |
| templates | | are needed. |

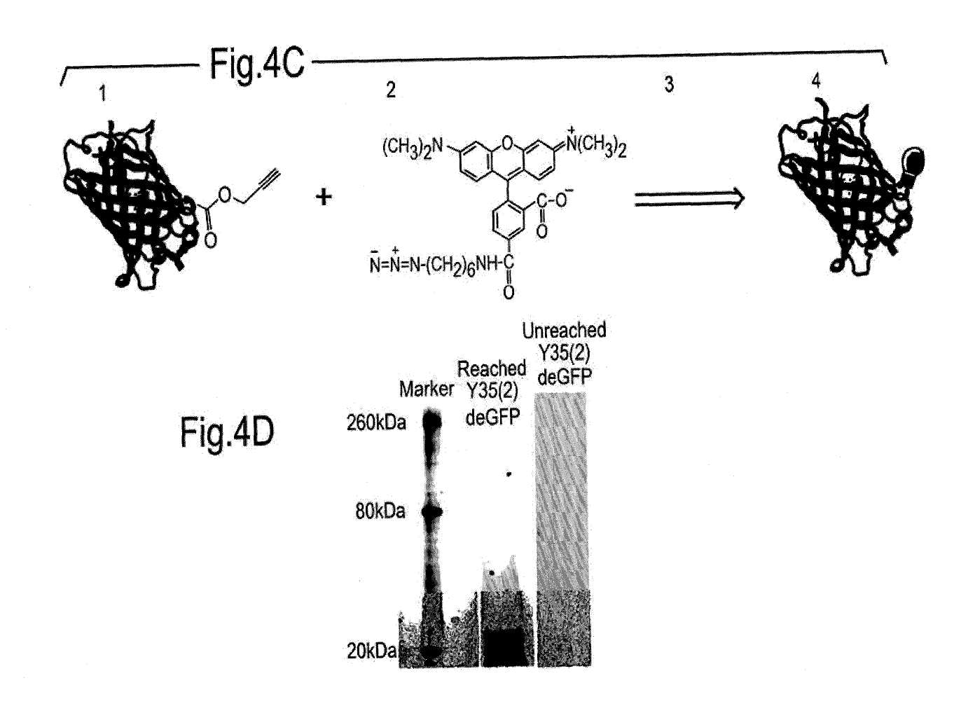

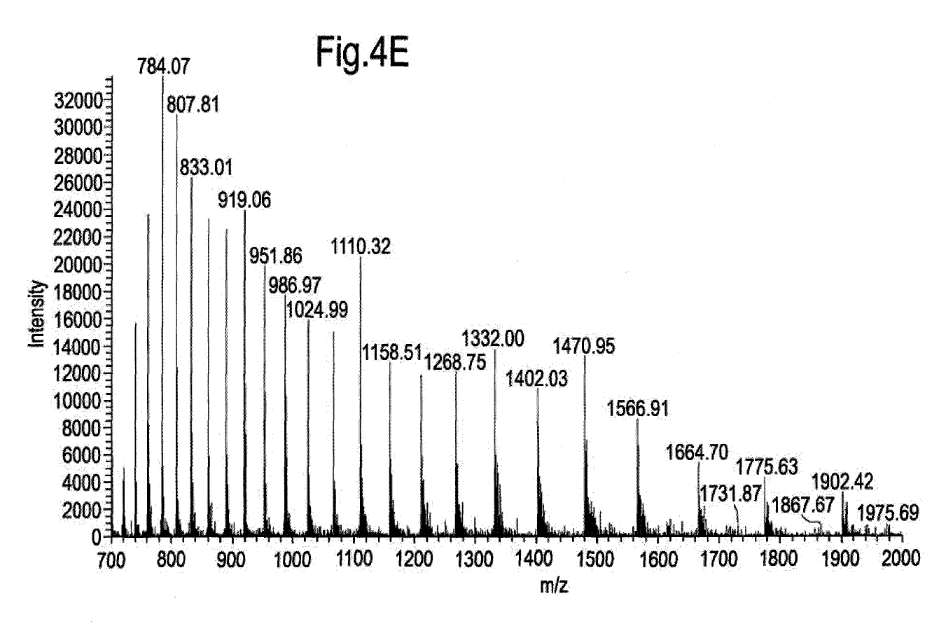

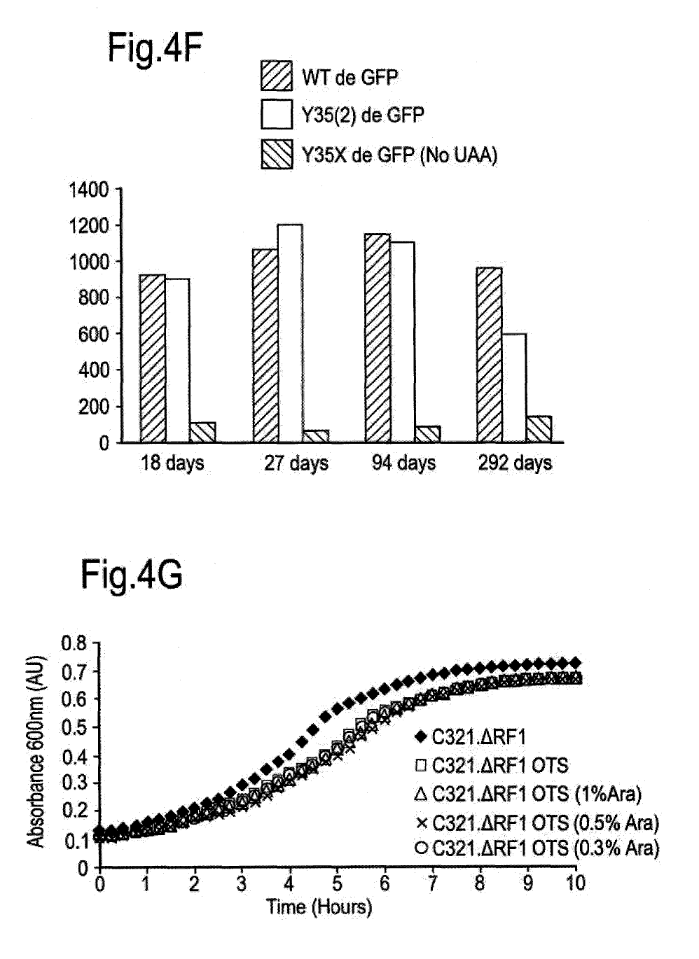

PrK Incorporation was confirmed via mass spectrometry and a “click” reaction. Using electrospray ionization-mass spectrometry (ESI-MS), the mass of WT deGFP expressed as described was compared to that of the deGFP containing PrK (Y35X) (compare FIG. 4A versus FIG. 4B), which provides for the verification of the correct incorporation of UAA and excludes the possibility of a ribosomal read-through (i.e. background suppression) of the system when the protein was expressed in the presence of PrK.

In order to confirm the incorporation of the bioorthogonal propargyl functional group into deGFP, the cell-free-produced and purified deGFP was compared to a catalyzed Huisgen 1,3-cycloaddition (“click”) reaction. In this reaction, the fluorescent Tamra-azide dye (shown in FIG. 4C) was ligated to deGFP site specifically where PrK had been incorporated (FIG. 4 C). The results of this reaction were observed using SDS-PAGE in gel fluorescence analysis using specific filters for the Tamra fluorophore (excitation: 520 nm; emission 575 nm); (FIG. 4D).

The MS results show a mass difference of 47.1 Daltons, a value that is in a good agreement with the calculated mass difference between deGFP containing PrK and WT deGFP with a tyrosine at position 35, which is a difference of 47 Daltons. Furthermore, only a single peak which corresponds to a total mass of 26,669.6±2.2 Daltons was observed upon ESI-MS analysis of purified deGFP Y35X (a value that coincides well with the calculated mass of 26,670 Daltons for our mutant protein), confirming that no background suppression had occurred in the presence of the UAA. These results imply that the presence of PrK in the reaction led to no detectable background suppression by natural amino acids instead of PrK (detailed ESI-MS results, FIG. 4E). The “click” reaction results (FIG. 4D) clearly show a fluorescent band corresponding to ca. 27 kDa, a value in agreement with the calculated molecular mass of deGFP of 26.6 kDa, including an added Tamra-azide group which elevates its mass to ca. 27 kDa.

The possibility of detecting GFP fluorescence (excitation: 485 nm; emission: 525 nm) was excluded by a control experiment where Y35PrK deGFP that did not undergo “a click” reaction was checked for Tamra fluorescence. No fluorescence was observed under the same conditions used for the imaging of the gel shown in FIG. 4D; right lane. Lastly, when protein expression was attempted in the absence of PrK followed by an attempted “click” reaction using Tamra-azide, Tamra fluorescence was not observed (Data not shown). Thus the systems of this invention demonstrated PrK being the only incorporated amino acid in response to the UAG stop codon.

To assess system stability, a series of experimental repeats of three CFPS reactions were carried over the course of 3 months. Reactions using 3 different batches of plasmids, multiple aliquots of the “all inclusive” genetically expanded extracts containing the pyl OTS and multiple aliquots of the reaction enhancement buffer and results are presented in FIG. 3.

As is evident from FIG. 3, the CFPS of wild type deGFP was relatively stable with standard deviation error of ±12% between 14 different experiments. The CFPS of Y35X deGFP with 1 mM of Propargyl lysine (i.e. genetically expanded CFPS) was equally stable with standard deviation error of ±11% between 8 different reactions. The p-VALUE shows no significant difference between the values of the W.T and the genetically expended CFPS of deGFP.

Taken together, the results show that seamless expansion efficiency was obtained. The negative control reactions (Y35X deGFP with no UAA added) showed consistency and negligible expression levels (As explained earlier, the expression level of this negative is not 0 because of auto-fluorescence and ribosomal read-through).

From the latter results, we conclude that genetically expanded CFPS is stable.

Thus, the stability of the systems of this invention is significantly better than any other exogenous OTS system and represents only a starting point to anticipated greater stability yields with time.

To test the long storage effects on the genetically expanded CFPS, reactions were carried out at different time points and up to 10 months after the preparation of the lysate and buffer (FIG. 4F). After ˜10 months the system still had stable yields (ca. 85% of average yields) but suppression efficiency was reduced to ca. 60%. Leading us to conclude the genetically expanded lysate is stable for at least 3 month, after which the decay in suppression efficiency slowly becomes significant.

Since amber suppression could have toxic effects on the viability of E. coli strains, the effects in terms of toxicity of the OTS plasmid (pEVOL MbPylRS/PylT) on the C321.ΔPrfA strain were tested (FIG. 4G). From the results it is clear that the growth curves of all OTS transformed bacteria are essentially identical in all induction levels, and they are similar to the growth of the original untransformed C321.ΔPrfA strain.

Small negative differences in the growth curves between the transformed and the original strains are evident but inconsequential and likely as a result of the energy/resources spent by transformed bacteria in order to over express the plasmid genes and the need to replicate it in high numbers.

Thus, no noticeable toxicity was caused by the pEVOL-Pyl OTS to the C321.ΔPrfA bacterial strain. Thus, C321.ΔPrfA, having all the TAG instances removed does not suffer any handicap from amber (TAG) suppressors.

### Example 2

### Effective Cell Free Protein Synthesis Incorporating a Non-Natural Amino Acid: Validation in Two Enzyme Assays

In order to demonstrate successful incorporation of the non-natural Propargyl-Lysine (UAA) amino acid site specifically into a target protein, which is not a reporter system, activity assays for proteins prepared using the cell free protein synthesis systems of this invention were investigated. Toward this end, the target proteins: Zymomonas mobilis alcohol dehydrogenase II enzyme (ADHII) and the E. coli copper efflux oxidase (CueO) enzyme were synthesized via the cell free protein synthesis systems of this invention incorporating the non-natural amino acid Propargyl-Lysine.

In order to demonstrate that genetically expanded CFPS produces correctly folded and active enzymes, the system was validated using two enzymes. Expression of the enzymes Zymomonas mobilis Alcohol dehydrogenase (ZmADH)—a 382 amino acids enzyme dimer and Copper efflux oxidase—a 488 residue enzyme was undertaken via the methods of this invention. The enzymes were also mutated to contain a single amber mutation (as described in Table 1), as well. Following the genetically expanded CFPS with the addition of 1 mM Propargyl-Lysine the reaction mixture was then probed for specific activity of the thus translated proteins by the methods described hereinabove.

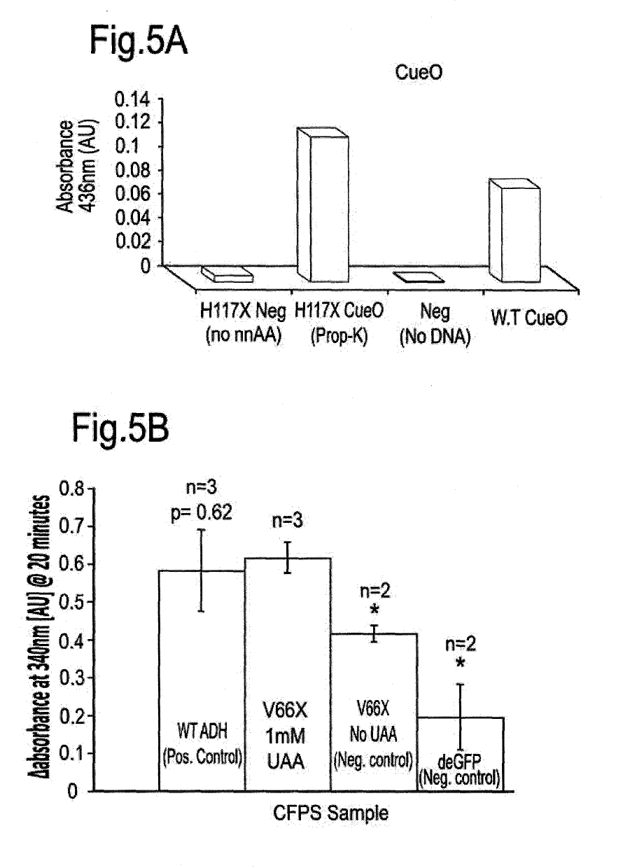

FIG. 5A plots the absorbance as a function of protein activity for wild type CueO and H117X CueO (Prop-K) containing the non-natural amino acid, at comparable levels, in comparison to negative controls containing no DNA or no non-natural amino acid, respectively. E. coli CueO produced in C321.Δ PrfA pEVOL-Pyl OTS CFPS was probed for its activity, in comparison to wild type E. coli CueO (i.e. No amber mutation—positive control), H117X E. coli CueO (Genetically expanded reaction with 1 mM of Propargyl-Lysine exogenously added) and H117X E. coli CueO with no UAA added (negative control). FIG. 5B plots ZmADH activity as a function of absorbance at the indicated wavelength in the C321.Δ PrfA pEVOL-Pyl OTS CFPS produced system versus wild type (WT) ZmADH (i.e. No amber mutation—positive control), V66X ZmADH (Genetically expanded reaction with 1 mM of Propargyl-Lysine exogenously added) and V66X ZmADH with no UAA added (negative control). Strains were prepared and alcohol dehydrogenase activity was measured as described The assay was carried directly on the reaction mixture without any purification steps. The results compare activity (Quantified by NADH formation measured as 340 nm absorbance). “n” is the number of reaction samples tested. ANOVA test was conducted comparing between V66X ZmADH and both the negative and positive controls. Pval<0.01 marked as **

As is readily seen in the figure, ADH activity was fully functional in the C321.Δ PrfA pEVOL-Pyl OTS CFPS produced system, despite the incorporation of the non-natural amino acid Propargyl-Lysine. Furthermore, no enzyme activity was evident in the absence of UAA, validating that the UAA was incorporated and that the system was fully functional, as expected.

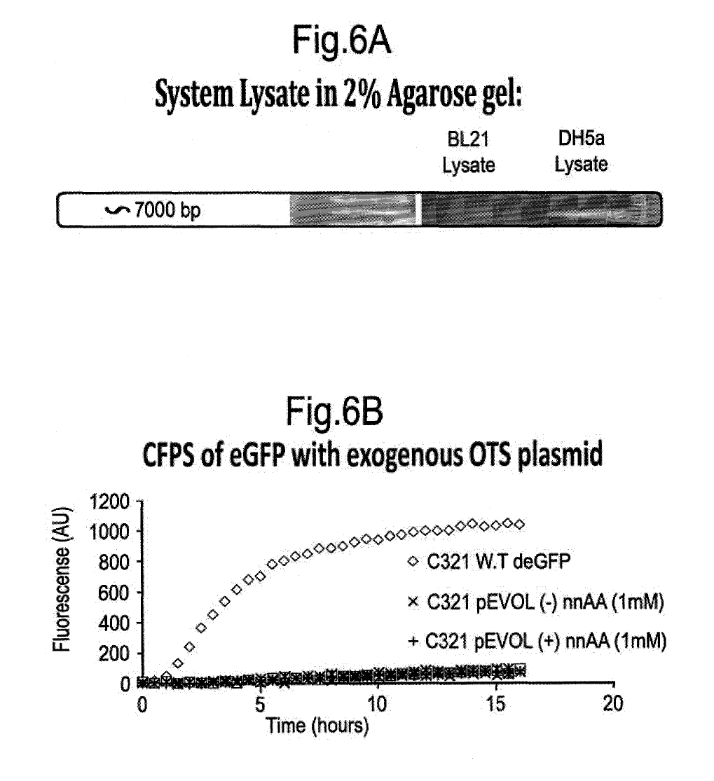

FIG. 6 demonstrates that the fate of the transformed OTS plasmid in the extract. During the preparation of the bacterial extract the chromosomal DNA of the source strain is removed after lysis using centrifugation.

It was of interest to assess whether the transformed plasmid, e.g. pEVOL pyl OTS is removed during centrifugation in order to address whether the presence of the plasmid could affect the CFPS reaction, for example by competing with the desired expression gene, thereby reducing yields.

Two cell extracts (Dh5alpha pEVOL-pyl and BL21 pEVOL-pyl) were therefore subjected to a mini prep protocol and sequentially run in an Agarose gel [FIG. 6A]. The results clearly show that the transformed OTS plasmid “survived” the cell extract process and is present in the cell extracts used for the genetically expanded CFPS.

The effect of the remnant transformed plasmids on the CFPS system was also evaluated.

In order to assess any potential effects contributing to a yield increase in the enhanced protein due to the co-production of o-tRNA during the CFPS reaction, a cell extract from the C321.ΔPrfA strain with no transformed plasmid was produced, and exogenous pEVOL-pyl OTS and the deGFP expression plasmid were added together in the CFPS reaction.

If the OTS plasmid in the final CFPS mixture is sufficient for genetically expanded reaction or is key to the reaction yields, similar results were expected, in comparison to the “all inclusive” system.

Surprisingly, the results were negative; i.e. no noticeable amount of enhanced protein was produced in the exogenously added OTS system (FIG. 6B).

Thus, a genetically expanded cell free protein synthesis system was developed and validated. Multiple bacterial systems and orthogonal pairs were employed and validated, showing the broad applicability of the systems, methods and kits of this invention.

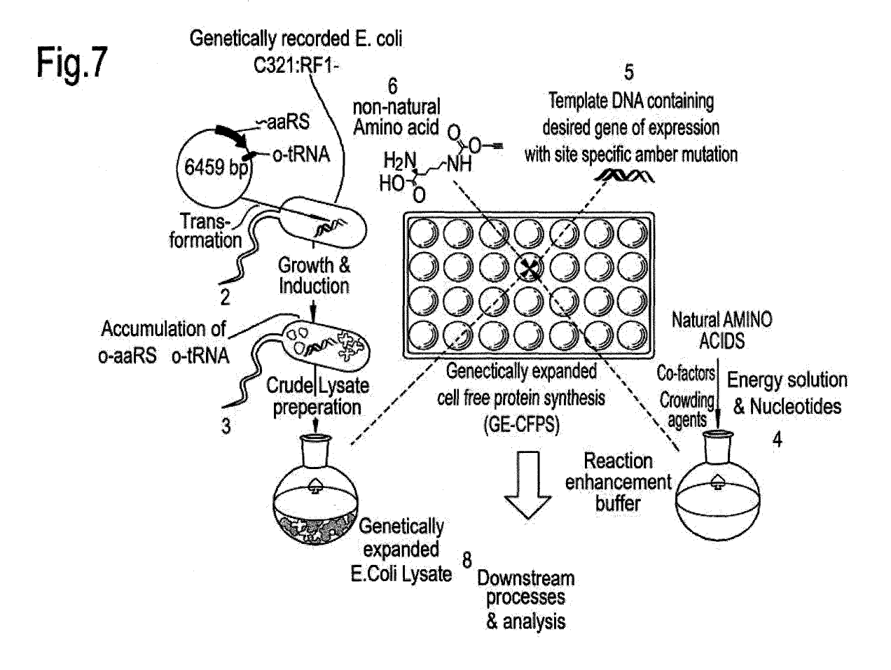

FIG. 7 schematically depicts a genetically expanded cell free protein synthesis method of this invention. The following steps in the figure are highlighted: step 1) Transformation of pEVOL PylRS/PylT OTS into C321:RF1− strain. 2) Growth phase, induction at O.D600 nm of 0.5-0.7 and harvest at Early-Mid Log phase of O.D600 nm 1.5-2. 3) Crude lysate extract preparation (can be aliquoted and stored long periods [>1 year]). 4) Preparation of Reaction enhancement buffer (see materials and methods) containing all natural amino acids, Co factors, Crowding Agents (PEG) and energy containing molecules. This buffer could be aliquoted and stored at −80 for very long periods 5) Template DNA, containing either linear PCR products or plasmids—in this work the pBEST plasmid was used. Desired expressed proteins were sub-cloned under the regulation of a mutant Lambda Bacteriophage promoter. The gene of interest was pre-mutated to contain an amber (TAG) mutation at the UAA incorporation site. 6) The relevant UAA, compatible with the OTS, must be added to the reaction mixture to enable incorporation. 7) All 4 components 3, 4, 5, 6 are mixed and incubated for 5-8 (until saturation). 8) The expressed “enhanced” protein (containing UAA) can be rapidly analyzed and used.

To date, there has not been any successful incorporation of Pyrro-Lysine in vitro and the cell free protein synthesis systems of this invention have surprisingly achieved the same in substantial yields showing marked activity.

### Example 3

### Effective Cell Free Protein Synthesis Incorporating a Second Non-Natural Amino Acid: Δ-Thio-ε-Boc-Lysine (TBL)

In order to demonstrate successful incorporation of another non-natural amino acid, the non-natural Δ-Thio-ε-Boc-Lysine (UAA) amino acid was incorporated site specifically into a target protein. Toward this end, the EGFP variant that optimized for cell-free protein synthesis (deGFP) was utilized, which was expressed under the control of the OR2-OR1-PR promoter, similar to the method as described in Example 1.

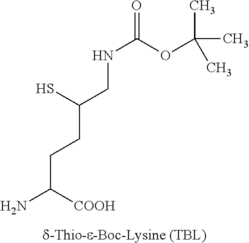 [SMILES: CC(C)(C)OC(=O)NCC(S)CCC(N)C(=O)O]

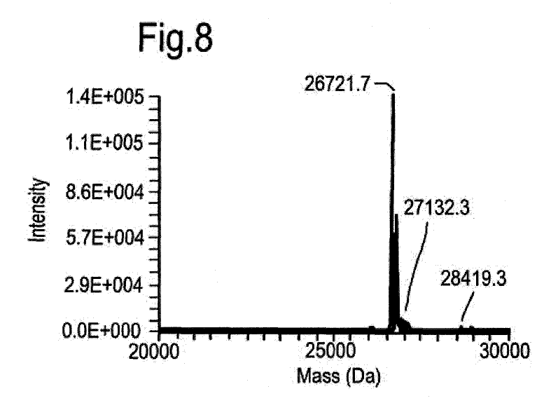

Following preparation of the cell free protein synthesis system as described above utilizing the pEVOL derivative plasmids expressing the OTS in the E. coli strain C321.ΔPrfA, cell free protein synthesis was carried out in the presence of wild type Mm-pyl synthetase and EPI mass spectrometry conducted. FIG. 8 demonstrates the results of the EPI mass spectrometry showing a clear peak at 26721.7 Dalton, which correlates within 1 Dalton to the calculated mass of a deGFP containing TBL at position 35 (FIG. 8).

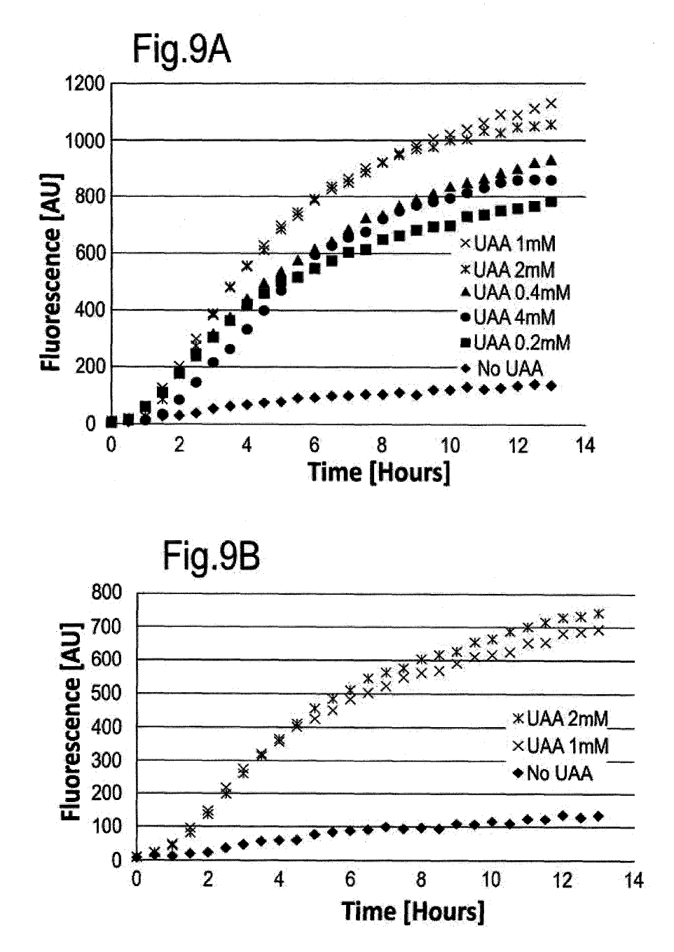

The cell free protein synthesis system was evaluated for the incorporation of the two pyrrolysine derivatives into the model protein, deGFP, as well, with mutagenesis of the Y35X codon position conducted (where X denotes TAG). At t=0, the pBEST_deGFP plasmid was added to the different reaction mixtures. To each mixture, a different concentration of UAA; either N^{ε}-Propargyl-l-lysine or N^{ε}-Boc-l-lysine was added (both are known to be recognized by the Pyl-OTS). As a negative control, no UAA was added, as a positive control wild-type deGFP(deGFP without amber mutations) was used. The reactions were incubated and the subsequent fluorescence of the deGFP produced was monitored in real time using a fluorescence plate reader. FIGS. 9A and 9B show the increase in fluorescence resulting from the expression of a full length deGFP containing N^{ε}-Propargyl-l-lysine or N^{ε}-Boc-l-lysine, respectively.

The results show the UAA concentration-dependence of deGFPdeGFP fluorescence, confirming that the UAA and orthogonal tRNA specifically recognized by PylRS and ribosomal translation of the TAG codon as a sense codon was enabled. Moreover, in this system, expression of UAA-incorporated deGFP is similar to the wild type (WT) deGFP expression rate.

Western blot analysis with anti-GFP immunoblotting confirmed the respective UAA incorporation, as well (data not shown).

Furthermore, incorporation of the TBL unnatural amino acid, in particular, should enable site specific ligation of any two proteins in a practical manner, not available to date. One application, for example, is the means to provide site specific ubiquitinylation of proteins.

In some aspects, the procedure provided hereinabove allows for the incorporation of “ubiquitin codes” to promote its incorporation, i.e. ligation between ubiquitins and a substrate protein.

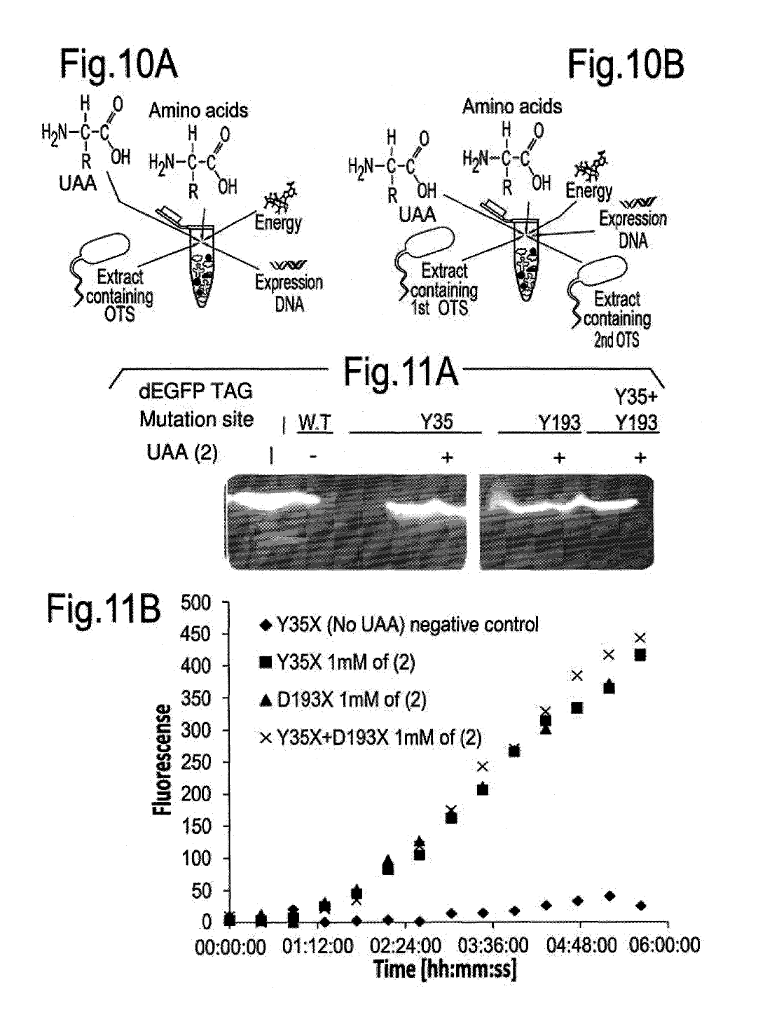

FIG. 10A schematically depicts a process for introducing any ubiquitin code (polyubiquitin) using the genetically expanded and endogenous cell free protein synthesis methods as herein described. First the unnatural amino acid Δ-Thio-ε-Boc-Lysine is incorporated into Ubiquitin proteins genetically fused to Intein and Chitin binding domain proteins. The Chitin Binding domain is used in tandem with a chitin column for purification purposes (i.e. separating the desired ubiquitin construct from the reaction mixture). Next, the intein protein can be cleaved—using a reducing agent (MESNA). Once the intein is cleaved a thio-ester reactive group will be formed in the N-terminus of the Ubiquitin. The thio-ester moiety can then undergo native chemical ligation with another ubiquitin protein construct, synthesized separately and still haven't gone through intein cleavage but the Boc protection group is removed (using strong acid) from the site specifically incorporated Δ-Thio-ε-Boc-Lysine. Once the two proteins are mixed together the Thio-ester moiety and the deprotected Δ-Thio-Lysine can undergo site specific native chemical ligation using published procedures. This methodology can be repeated to create essentially any permutation or sequence of ubiquitins. Thus this methodology enables the fabrication of any ubiquitin code desired by the user.

FIG. 10B schematically depicts the next stage, whereby after obtaining the sought after ubiquitin code as described in FIG. 18A, it is necessary to ligate the fabricated code to a substrate protein in order to actualize the function of the code. In this aspect, it is considered to ligate the fabricated code to a GFP protein, as a model protein. Ligation to such protein will enable the user to investigate the different meanings of any ubiquiting code ligated to the GFP by cell transfection of the ubiquitinated GFP and observation of its behavior once transfected.

The ligation between the ubiquitin code and the substrate protein uses similar methodology as the fabrication of the Ubiquitin code. Through native chemical ligation between the site specific incorporated (and deprotected) Δ-Thio-ε-Boc-Lysine of the synthesized substrate protein and in the N-terminus thio-ester of the ubiquitin chain (created by the intein cleavage).

### Example 4

### Effective Cell Free Protein Synthesis Incorporating Multiple Unnatural Amino Acids (UAAs)

### Additional Materials and Methods

### Reagents:

Propargyl-L-lysine (PrK) was dissolved in water. A p-azido-L-phenylalanine (pAZF) solution was prepared, as well. pAZF was dissolved in 1 mL NaOH 1M for a final concentration of 100 mM pAZF. Following that, 100 uL of pAZF 100 mM were acidified with 96 uL HCl 1.04M and was added to HEPES buffer 250 mM to a final volume of 1 mL, resulting in a final concentration of 10 mM pAZF.

### Orthogonal Translation System (OTS) Anti-Codon Modification:

PrK incorporation in response to a TAG stop codon is described hereinabove, using an OTS plasmid (pEVOL pylRS—containing the orthogonal pyrrolysyl tRNA synthetase and tRNA). pAZF incorporation in response to a TAA stop codon was achieved via the use of an OTS plasmid: pEVOL pAZF (Addgene #31186), which plasmid contains an orthogonal tyrosyl synthetase mutant [pAZF-RS] and tRNA, The tRNA in the pAZF OTS plasmid underwent site-directed mutagenesis of the anti-codon from CTA into TTA, this modification provided for the avoidance of any cross reactivity between the two systems.

### Expression Plasmids:

The reporter protein deGFP and the expression plasmid variants of the pBEST-OR2-OR1-Pr-UTR1-deGFP-T500 were used (Addgene #40019). Before mutating different sites of the deGFP gene for UAAs incorporation, the termination codons of all of the proteins (deGFP and antibiotic resistance) were replaced (i.e. TAA was replaced with TGA), as sequential use of a TAA stop codon for pAZF incorporation would be in conflict, and use of the TGA stop codon, allows for release factor 2 to end the translation process as needed. Once the termination codons were altered to TGA, the plasmid functions as the WT in this system, and deGFP genes with following mutated sites are produced: Y35TAA, Y35TAG, D193TAA, D193TAG, Y35TAA D193TAG and Y35TAG D193TAA. Site directed mutagenesis was utilized to introduce the changes to the plasmids as described.

### Cell Extracts Preparation:

Two extracts were created. A first extract, containing the pyrrolysyl synthetase and tRNA (PylRS/tRNA_{Pyl}) OTS as a result of transformation of pEVOL pylRS into the wanted bacteria, was prepared. A second extract, containing the tyrosyl derivative synthetase and tRNA (pAZF-RS/tRNA_{Tyr}) OTS by transformation of a different plasmid, the pEVOL pAZF, into the bacteria of interest, was also prepared. In this case, the C321.ΔprfA bacterial strain (release factor 1 knockout and 321 TAG sites changed into TAA) was used to prepare both extracts, with the cell extract preparation protocol identical to that described hereinabove. Other bacterial strains can be used, as well.

### Cell Free Protein Synthesis (CFPS):

The CFPS reaction volume, temperature, buffer composition, buffer amount per reaction, expression plasmid amount, incubation time and fluorescence measurements, etc. are comparable to those described in the CFPS assays hereinabove. The UAAs (pAZF/PrK) were added to a final concentration of 1 mM, as needed, in terms of the expression plasmid and controls used. The CFPS reaction consisted of 33% E. coli extracts, as above, however two different lysates were used to make up the extract. The two extracts were added to the reaction master mix in a 1:1 ratio. Since both extracts were from the same strain of bacteria, the strain possessed the same native translation components, and mixing the two lysates together provided for the presence of two different OTSs at once and allowed the CFPS to incorporate natural amino acids, as well as pAZF and PrK, as a function of the respective corresponding codons.

Purification of the reporter deGFP protein for LC-MS validation of incorporation of UAAs and for click reaction, nickel affinity chromatography purification of 6×his-tagged deGFP was performed. 250 μL of CFPS reaction mixture was incubated overnight at 29° C. to produce either deGFP Y35X (N-terminal 6×his tag) or WT deGFP (N-terminal 6×his tag). The reaction mixture was then diluted with 3 volumes of PB buffer (50 mM PB pH 8, 0.3 mM NaCl and 10 mM imidazole) and added to a nickel-bead column (Novagen, Madison, Wis.). Wash (50 mM imidazole) and elution (250 mM imidazole) steps were conducted according to the manufacturer's instructions. The protein-containing eluted fraction was concentrated using a Vivaspin 10 kDa cutoff concentrator (Sartorius, Göttingen, Germany) The resulting concentrated fraction was analyzed by LC-MS (Finnigan Surveyor Autosample Plus/LCQ Fleet, Thermo Scientific, Waltham, Mass.).

“Click” reaction. The deGFP containing both PrK and pAZF were labeled using the Cu(I) catalyzed azide-alkyne cycloaddition reaction (CuAAC). Protein sample was resuspended in 0.1M PB pH=7.5. Two types of reactions were performed separately: one “click” reaction for the conjugation of a marker to PrK, and the second “click” reaction for the conjugation of a different marker to pAZF. For the conjugation to PrK, Tetramethylrhodamine-Azide (TAMRA-Az) (Sigma) was added to a concentration of 100 μM. THPTA, Sodium ascorbate and CuCl₂ were added to final concentrations of 400 μM, 2.5 mM and 200 μM, respectively. The reaction mixture was incubated at room temperature for from 3-12 hours. 20 μL sample from the mixture was diluted with 4×SDS sample buffer and kept for 10 min at 70° C., after which it was loaded and run on a 12% SDS-PAGE gel. Labeled proteins were visualized in-gel using ImageQuant LAS 4000 imager (Fujifilm, Tokyo, Japan), in fluorescence mode. For the conjugation to pAZF, ATTO-alkyne (sigma) was added to a concentration of 100 μM. THPTA, Sodium ascorbate and CuCl₂ were added to final concentrations of 400 μM, 2.5 mM and 200 μM, respectively. The reaction mixture was incubated at room temperature for from 3-12 hours. 20 μL sample from the mixture was diluted with 4×SDS sample buffer and kept for 10 min at 70° C., after which it was loaded and run on a 12% SDS-PAGE gel. Labeled proteins were visualized in-gel using ImageQuant LAS 4000 imager (Fujifilm, Tokyo, Japan), in fluorescence mode.

### Results

The ability to create an endogenous system for cell free protein synthesis (CFPS) incorporating two different unnatural amino acids (UAAs) provides a flexible expression platform.

Incorporation of multiple Propargyl-lysine UAAs into various sites in deGFP can be accomplished by incorporating, for example, N^{ε}-Propargyl-l-lysine simultaneously into two deGFP sites, Y35 and D193 (FIG. 11A). Western blot results confirmed deGFP expression with no visible reduction in deGFP expression levels when the 2 TAG mutations were introduced into the same gene. Quantitative fluorescence results (FIG. 11B) support this finding, as well. Although some expression theoretically may be attributable to low level TAG ribosomal read-through, the efficiency and enhanced expression as evidenced in this system supports the added advantage in terms of the unique methods for multiple site-specific UAA incorporations, as herein described.

The ability to create an endogenous system for cell free protein synthesis (CFPS) incorporating two different unnatural amino acids (UAAs) in response to two different stop codons provides an enormously flexible platform with many applications and therefore was pursued, as well.

To develop such a system, first, an endogenous in-vitro system for the incorporation of propargyl-L-lysine or N-boc-L-lysine in response to an amber stop codon (TAG) was created as described hereinabove. Methanosarcinamazei (Mm) pyrrolysyl-tRNA synthetase and pyrrolysyl tRNA (PylRS/tRNA^{Pyl}) were cloned into a genomically recoded E. coli prior to the lysis phase and a CFPS lysate (activated cell extract) was produced from this modified E. coli.

An endogenous CFPS system for the incorporation of p-azido-L-phenylalanine in response to ochre stop codon (TAA) was also created similar to the CFPS system for incorporation of the UAA in response to an amber stop codon. Toward this end, methanocaldococcusjannaschii (Mj) tyrosyl-tRNA synthetase and tyrosyl-tRNA (TyrRS/tRNA^{Tyr}) were cloned into the genomically recoded E. coli strain referred to hereinabove prior to the lysis phase and a CFPS lysate (activated cell extract) was produced from this modified E. coli as well.

Separately, each of the two systems can synthesize a protein with the specified UAA incorporated at a specific site. Moreover, each system can synthesize, in theory, any protein with any of the substrate UAAs of the mentioned amino-acyl tRNA synthetase.

In one aspect of the invention, it was considered that combining the two lysate systems allows for the incorporation of two different UAAs in response to two different stop codons in-vitro.

Mixing two lysates and adding the energy solution, natural amino acids, the two UAAs and the expression plasmid allows for the creation of a system able to incorporate propargyl-L-lysine in response to the amber stop codon (TAG) and p-azido-L-phenylalanine in response to the ochre stop codon (TAA) in deGFP (FIG. 10A and FIG. 10B). In all cell free protein synthesis systems (CFPS), there is a need for the exogenous supply of energy, natural amino acids, an expression plasmid and a bacterial extract. For the genetically expanded CFPS, the expression plasmid must have the proper mutations, the relevant UAAs to be added and the bacterial extract containing a relevant OTS. When incorporating one UAA, a single type of extract, containing OTS, is needed, but when incorporating two different UAAs, two extracts (but in some embodiments, using the same bacterial strain) containing two different OTSs must be added (See FIG. 10A versus 10B, respectively).

In order to validate the ability to express two different UAAs in response to two different stop codons in a cell free system, a deGFP reporter system was utilized. deGFP was cloned into the pBEST expression plasmid (as described hereinabove). In order to work with deGFP, while simultaneously suppressing TAG and TAA stop codons, the termination codon for the protein translation was changed from an ochre (TAA) into an opal stop codon (TGA). Different variants were created, by replacing the tyrosine amino acid at site 35 and the aspartic acid amino acid at site 193 with the two stop codons for incorporation (TAG and TAA). Different variants were created to test all different conditions:

1. Y35TAG

2. Y35TAA

3. D193TAG

4. D193TAA

5. Y35TAG D193TAA

6. Y35TAA D193TAG

The incorporation of two different UAAs in response to two different stop codons, was achieved by mixing the two lysates and two such constructs were created, as described for (5) and (6) hereinabove.

Fluorescence was monitored as an indicator of the incorporation of the two different UAAs and various controls.

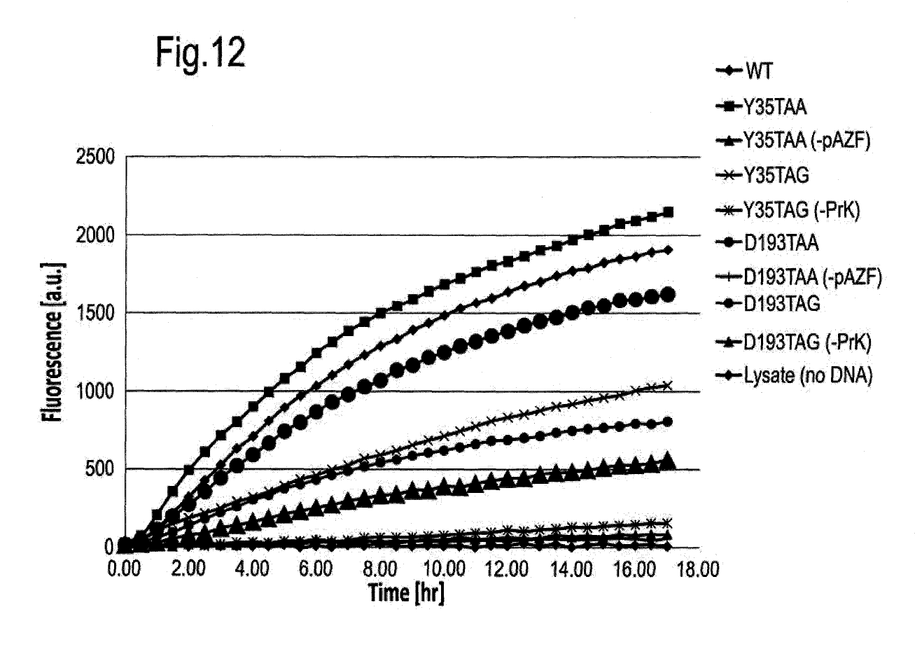

FIG. 12 depicts the results of mixed in vitro expression of deGFP from C321 pEVOL pylRS TAG lysate+C321 pEVOL pAZF TAA lysate. FIG. 12 depicts the results of a cell free protein synthesis reaction prepared as above, combining two cell lysates as described. In the figure, each OTS is demonstrated to be capable of performing independently, and the functionality of each OTS is seen, as well, thus single UAA incorporation can proceed, even in a combined system where 2 UAAs are present. The WT represents a deGFP gene with no nonsense (stop codons) mutations for UAAs incorporation, therefore unable to facilitate such an incorporation and serving as a negative control.

Other controls include CFPS with the indicated strains having the same genes but without the different UAAs, demonstrating a lack of incorporation of natural amino acids and incorporation of the respective UAA alone, within the target site (providing background levels).

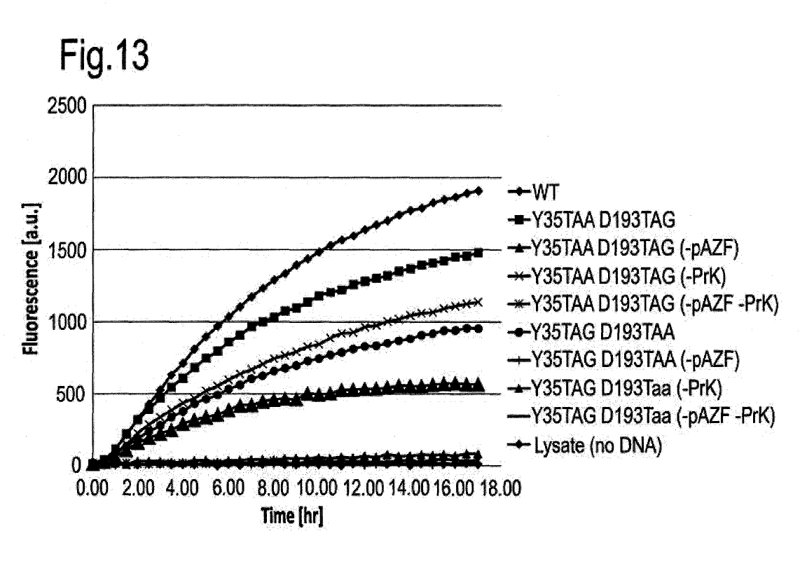

As is seen from the figure, greater fluorescence is observed with the Y35TAA than even the wild type, and D193TAA fluorescence is quite pronounced, as well. The Y35TAG, D193TAA and D193TAG constructs, all performed better than the same constructs, when PrK or pAZF were absent from the expression system. FIG. 13 depicts the results of the mixed in vitro expression of deGFP from C321 pEVOL pylRS TAG lysate+C321 pEVOL pAZF TAA lysate where expression of the two non-natural amino acids in a cell free in vitro system was assessed. FIG. 13 depicts the results of a cell free protein synthesis reaction prepared as above, combining two cell lysates as described. In the figure, the combined system where 2 UAAs are present is shown. The WT represents a deGFP gene with no nonsense (stop codons) mutations for UAAs incorporation, therefore unable to facilitate such an incorporation and serving as a negative control.

As is seen from the figure, greater fluorescence is observed with the wild type followed by Y35TAA D193TAG and Y35TAA D193TAG (−PrK), which is followed by Y35TAG D193TAA, and then Y35TAG D193TAA (−PrK). It is noted that lysate alone and Y35TAA D193TAG (−pAZF) did not give appreciable fluorescence.

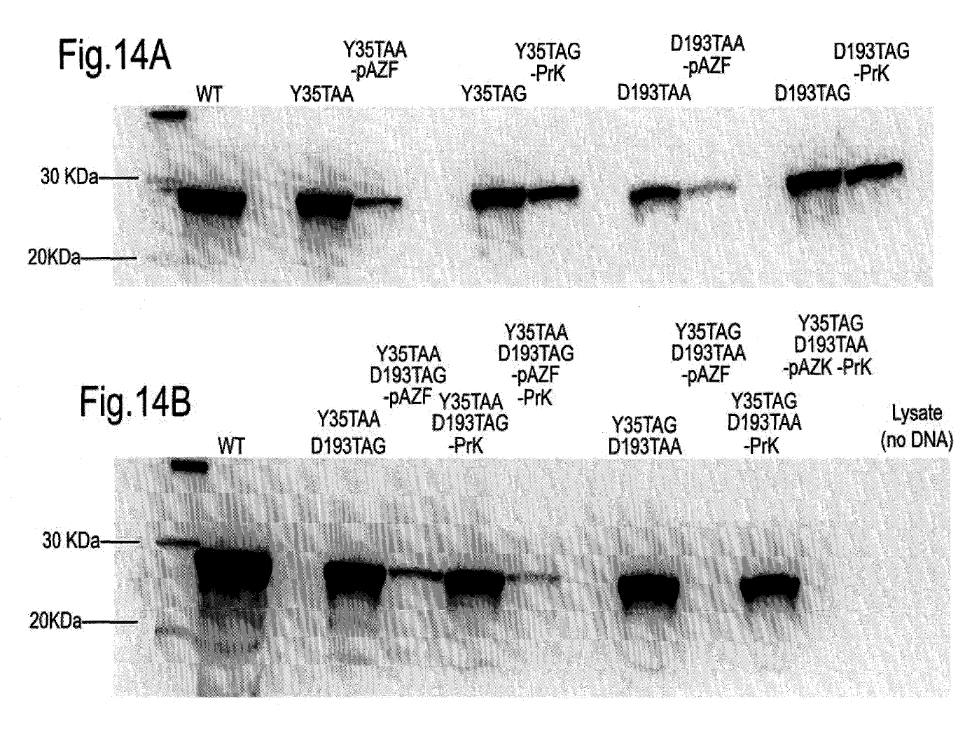

FIG. 14 provides the results for Western blot analysis probing using an anti-GFP antibody, validating the fluorescence measurement results in FIGS. 12 and 13. FIG. 14A parallels the results seen in FIG. 12 and FIG. 14B parallels the results seen in FIG. 13 in terms of expression levels of the in vitro expressed products.

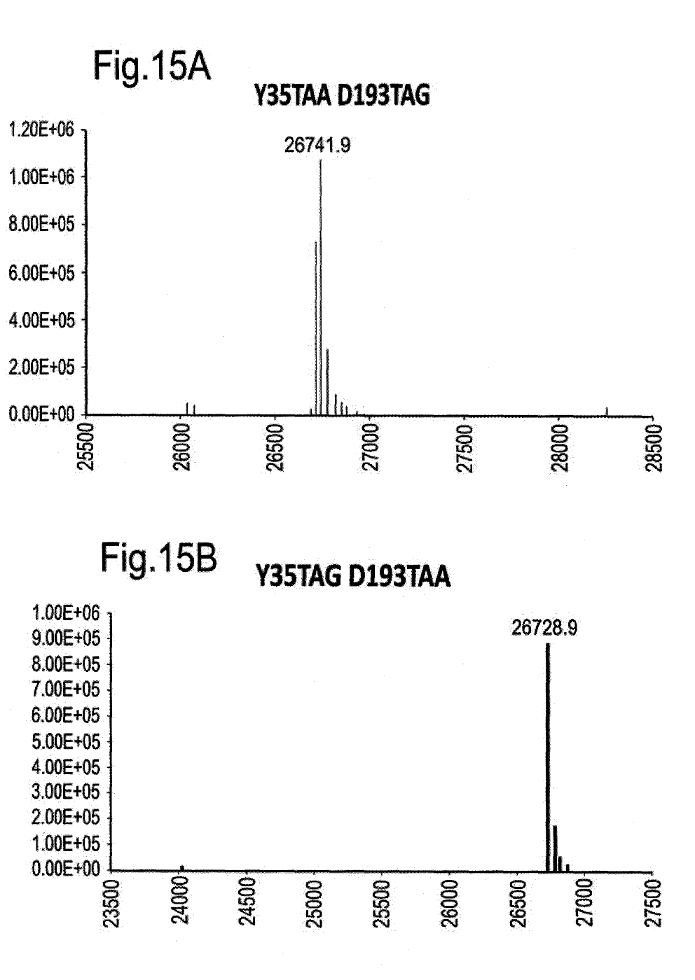

Protein identification was also achieved by mass spectrometry for the Y35TAA D193TAG and Y35TAG D193TAA products, respectively depicted in FIGS. 15A and 15B.

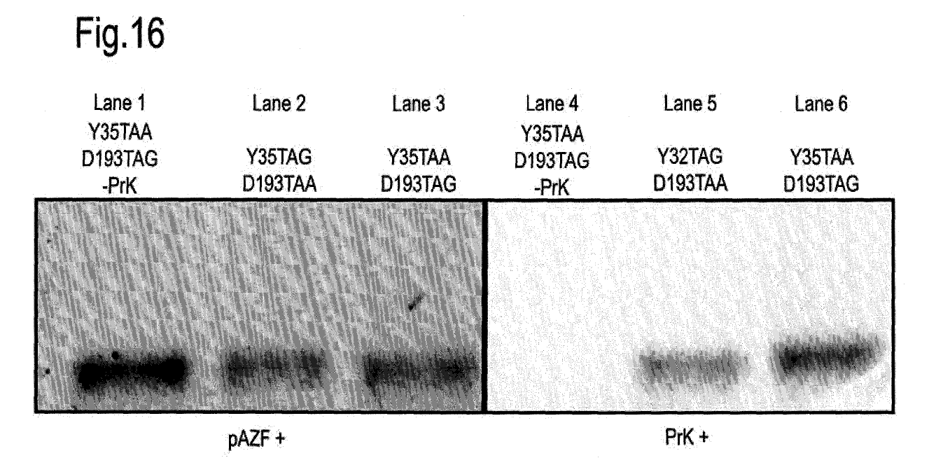

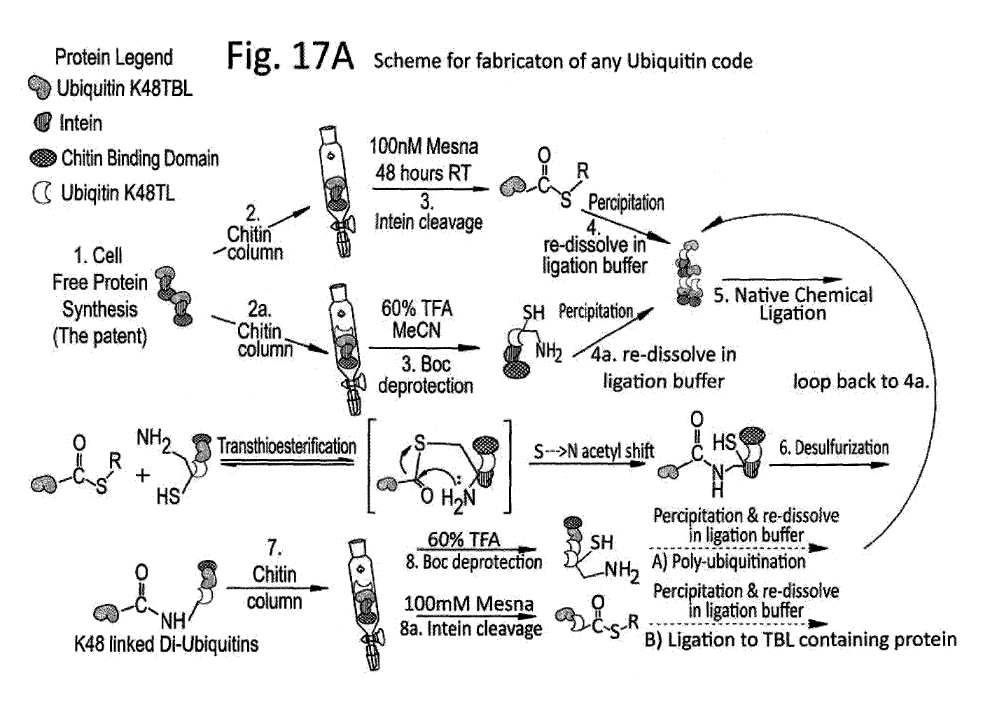

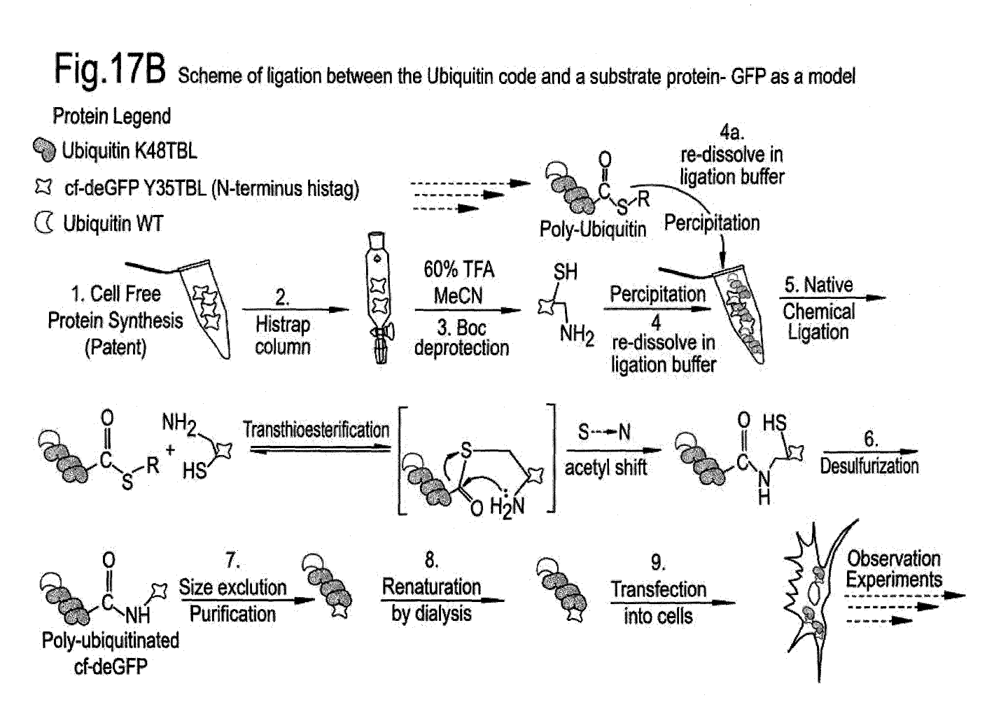

FIG. 16 depicts the results of further confirmation for the presence of the UAAs by click chemistry. After click chemistry, proteins underwent gel electrophoresis, which was later imaged using a fluorescence gel imager, the methods of which are described hereinabove. The same protein was tested for a covalent bond to Tamra-Az and ATTO-alkyne separately, such that the Tamra-Az conjugates to alkynes, such as the PrK, while the ATTO-alkyne conjugates to azides, such as the pAZF. As is readily seen in the figure, a prominent band for each double mutant is seen in FIG. 16, i.e. each double mutant can conjugate to both markers, indicating the incorporation of both of the UAAs. Lane 4, in which PrK was not provided in the cell free system, served as an indicator of the background fluorescence in this system (basal expression level).

Thus, using a combination of two different lysates in the cell free in vitro expression systems described, successful incorporation of two different UAAs in response to two different stop codons is enabled.

This invention demonstrates the successful incorporation of two different types of UAAs into a single protein, using an endogenous in-vitro system, for two different stop-codons suppression.

The system developed herein provides a unique means for greater protein manipulation, for example, by increasing the diversity of possible modifications introduced into such protein, including all the advantages known in the use of a CFPS system.

A variety of useful extensions of the systems and materials of this invention include FRET-based applications, cross-linking applications, ligation of cyclic proteins/peptides, addition of two different post translational modifications at once and others.

One of the many advantages to the systems and materials of this invention is the use of a CFPS system, which avoids the necessity for intact bacterial systems for expression, which in turn can result in interference by the bacterial genome.

For example, in introducing an OTS for stop codon suppression, in an intact bacterial in-vivo system, there is also the potential for suppression of TAG other than TAG suppression in the expression plasmid, which in turn could result in toxic results to the bacteria (manifesting, for example, as low protein yields).

An added advantage in the systems and materials of this invention is the use of the strain C321.ΔprfA, which provides for genetic code expansion without damaging the bacteria itself, with respect to TAG suppression.

Yet when multiple stop codons, as e.g. shown hereinabove, is attempted, with the use of two different stop codons, it is not possible to suppress both in a live in vivo system without causing toxicity to the bacteria. Thus there is a distinct advantage to cell free systems as herein described.

Surprisingly, higher yields were also obtained for the expressed product, in some cases above the wild type, as shown herein.

Additional advantages of the systems and materials of this invention include, for example, the ability to synthesize two different proteins at once, for example, with each incorporating a different UAA, and moreover, the expressed product can be conjugated to one another through the respective UAAs. In some embodiments, such conjugated products provide for the ability to create a complex of two proteins with different UAA permutations, providing an extremely versatile platform for many varied applications, as will be appreciated by the skilled artisan.

In another aspect, the materials and systems of this invention provide for testing drug activity, such as for example, antibiotics, against protein complexes. For example, and in some embodied aspects, it is possible to synthesize two different proteins at once, with each protein incorporating a different UAA, which two UAAs are capable of FRET. While the complex is intact, energy transfer between the two fluorophores occurs resulting in the energy transfer, however, when the drug interferes with complex formation/maintenance, then FRET is abolished and readily detected.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

In one embodiment of this invention, “about” refers to a quality wherein the means to satisfy a specific need is met, e.g., the size may be largely but not wholly that which is specified but it meets the specific need of cartilage repair at a site of cartilage repair. In one embodiment, “about” refers to being closely or approximate to, but not exactly. A small margin of error is present. This margin of error would not exceed plus or minus the same integer value. For instance, about 0.1 micrometers would mean no lower than 0 but no higher than 0.2. In some embodiments, the term “about” with regard to a reference value encompasses a deviation from the amount by no more than 5%, no more than 10% or no more than 20% either above or below the indicated value.

In the claims articles such as “a”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

1. A method for producing a rare amino acid- or non-natural amino acid-containing protein in a cell free protein synthesis system said method comprising:
expressing at least one orthogonal suppressor tRNA (o-tRNA)/aminoacyl-tRNA synthetase (aaRS) pair or derivatives thereof specific for incorporation of a rare amino acid- or non-natural amino acid in an E. coli organism;
preparing a lysate of said E. coli organism expressing said orthogonal suppressor tRNA (o-tRNA)/aminoacyl-tRNA synthetase (aaRS) pair; and
contacting said lysate with a template DNA containing a mutant gene in which at least one amino acid codon at a given site of the protein-encoding gene has been mutated into an amber or ochre mutation and further providing a cognate rare amino acid or non-natural amino acid and other factors necessary for protein synthesis;
wherein protein synthesis occurs following said contact to produce a protein containing said at least one rare amino acid or said non-natural amino acid.

2. The method according to claim 1, wherein said E. coli is genomically recoded to lack TAG codons in the genome and optionally to lack RF1.

3. The method according to claim 1, one or more of:
wherein said rare or non-natural amino acid is Propargyl-L-lysine and said aminoacyl-tRNA synthetase is pyrrolysyl-tRNA synthetase and the orthogonal tRNA is tRNA_{pyl}; or
wherein said rare or non-natural amino acid is N-Boc--Thio-L-lysine and said aminoacyl-tRNA synthetase; is pyrrolysyl-tRNA synthetase and the orthogonal tRNA is tRNA_{pyl}; or
wherein said rare or non-natural amino acid is p-azido-L-phenylalanine and said aminoacyl-tRNA synthetase is the tyrosyl-tRNA synthetase derivative Azido-L-Phenylalanine synthetase and the orthogonal tRNA is tRNA_{tyr}; or
wherein said rare or non-natural amino acid is N-boc-L-lysine and said aminoacyl-tRNA synthetase is pyrrolysyl-tRNA synthetase and the orthogonal tRNA is tRNA_{pyl}; or
wherein said rare amino acid is Δ-Thio-ε-Boc-Lysine and said aminoacyl-tRNA synthetase is pyrrolysyl-tRNA synthetase and the orthogonal tRNA is tRNA_{pyl}.

4. (canceled)

5. (canceled)

6. (canceled)

7. (canceled)

8. The method of claim 3, wherein said method further comprises the step of producing two rare amino acid- or non-natural amino acid-containing proteins in a cell free protein synthesis system by synthesizing two proteins containing said at least one rare amino acid or said non-natural amino acid.

9. The method of claim 3, wherein said method further comprises site-specific ligation of said two proteins.

10. The method according to claim 1, wherein said lysate is contacted with two different rare amino acids, which can be incorporated by the at least one orthogonal suppressor tRNA (o-tRNA)/aminoacyl-tRNA synthetase (aaRS) pair or derivatives thereof.

11. The method according to claim 3, wherein said two different rare amino acids are Para-Azido-L-phenylalanine and Propargyl-L-lysine.

12. The method according to claim 1, wherein said lysate is contacted with two different rare or non-natural amino acids, which can be incorporated by the at least one orthogonal suppressor tRNA (o-tRNA)/aminoacyl-tRNA synthetase (aaRS) pair or derivatives thereof.

13. The method according to claim 1, wherein said method comprises expressing two different orthogonal suppressor tRNA (o-tRNA)/aminoacyl-tRNA synthetase (aaRS) pairs or derivatives thereof, specific for incorporation of two different cognate rare amino acids- or non-natural amino acids in said E. coli organism.

14. The method of claim 9, wherein one of said two rare or non-natural amino acids is p-azido-L-phenylalanine and said aminoacyl-tRNA synthetase is the tyrosyl-tRNA synthetase derivative Azido-L-Phenylalanine synthetase.

15. The method of claim 9, wherein one of said two rare or non-natural amino acids is Propargyl-L-lysine and said aminoacyl-tRNA synthetase is pyrrolysyl-tRNA synthetase; or
wherein one of said two rare or non-natural amino acids is N-Boc--Thio-L-lysine and said aminoacyl-tRNA synthetase is pyrrolysyl-tRNA synthetase; or
wherein one of said two rare or non-natural amino acids is Δ-Thio-ε-Boc-Lysine and said aminoacyl-tRNA synthetase is pyrrolysyl-tRNA synthetase.

16. (canceled)

17. (canceled)

18. The method according to claim 1, wherein said protein containing at least one rare amino acid- or non-natural amino acid is a membrane-bound protein; or a secreted protein, or an enzyme, or an indicator protein.

19. (canceled)

20. (canceled)

21. (canceled)

22. A kit for producing at least one rare amino acid- or non-natural amino acid-containing protein in a cell free protein synthesis system said kit comprising:
at least one E. coli lysate formed from an E. coli organism expressing at least one orthogonal suppressor tRNA (o-tRNA)/aminoacyl-tRNA synthetase (aaRS) pair specific for incorporation of a rare amino acid- or non-natural amino acid in said E. coli organism;
reaction mix comprising UTP, GTP, ATP, CTP, NAD, tRNAs, CoA, 3-PGA, cAMP, Folic Acid, K-Glutamate, Mg-Glutamate, Spermidine, natural amino acids, cognate rare amino acids or non-natural amino acids, crowding reagents, pH buffer, and combinations thereof; and
optionally at least one template DNA containing a mutant gene in which at least one amino acid codon at a given site of the protein-encoding gene has been mutated into an amber or ochre mutation.

23. The kit according to claim 22, wherein said at least one E. coli lysate is formed from an E. coli organism genomically recoded to lack TAG codons in the genome, or lacking RF1 or a combination thereof.

24. The kit according to claim 10, wherein said rare amino acid is Pyrrolysine and said aminoacyl-tRNA synthetase is pyrrolysyl-tRNA synthetase.

25. The kit according to claim 22, wherein said rare or non-natural amino acid is Propargyl-L-lysine and said aminoacyl-tRNA synthetase is pyrrolysyl-tRNA synthetase; wherein one or more of:
said rare or non-natural amino acid is N-Boc--Thio-L-lysine and said aminoacyl-tRNA synthetase;
said rare or non-natural amino acid is pyrrolysyl-tRNA synthetase;
said rare or non-natural amino acid is p-azido-L-phenylalanine and said aminoacyl-tRNA synthetase;
said rare or non-natural amino acid is the tyrosyl-tRNA synthetase derivative Azido-L-Phenylalanine synthetase: is N-boc-L-lysine and said aminoacyl-tRNA synthetase;
said rare or non-natural amino acid is pyrrolysyl-tRNA synthetase;
said rare or non-natural amino acid is Δ-Thio-ε-Boc-Lysine and said aminoacyl-tRNA synthetase; and,
said rare or non-natural amino acid is pyrrolysyl-tRNA synthetase.

26. (canceled)

27. (canceled)

28. (canceled)

29. (canceled)

30. The kit according to claim 22, wherein said kit further comprises two different rare amino acids or non-natural amino acids.

31. The kit of claim 30, wherein said two different rare amino acids are Para-Azido-L-phenylalanine and Propargyl-L-lysine.

32. The kit of claim 30, wherein said kit comprises a first orthogonal suppressor tRNA (o-tRNA)/aminoacyl-tRNA synthetase (aaRS) pair or derivatives thereof and a second orthogonal suppressor tRNA (o-tRNA)/aminoacyl-tRNA synthetase (aaRS) pair or derivatives thereof.

33. The kit of claim 32, wherein said kit provides instructions for producing at least two rare amino acids- or non-natural amino acids-containing proteins in a cell free protein synthesis system.

34. The kit of claim 32, wherein one of said two rare or non-natural amino acids is Δ-Thio-ε-Boc-Lysine and said first aminoacyl-tRNA synthetase is pyrrolysyl-tRNA synthetase.

35. The kit of claim 34, wherein said kit further comprises reagents for the site-specific ligation of said at least two rare amino acids- or non-natural amino acids-containing proteins.

36. The kit of claim 32, wherein one of said two rare or non-natural amino acids is N-Boc--Thio-L-lysine and said first aminoacyl-tRNA synthetase is pyrrolysyl-tRNA synthetase.

37. The kit of claim 32, wherein one of said two rare or non-natural amino acids is p-azido-L-phenylalanine and said second aminoacyl-tRNA synthetase is the tyrosyl-tRNA synthetase derivative Azido-L-Phenylalanine synthetase.

38. The kit according to claim 30, wherein said template DNA containing a mutant gene in which at least one amino acid codon at a given site of the protein-encoding gene has been mutated into an amber or ochre mutation is provided as a linear template.

39. The kit according to claim 30, wherein said template DNA containing a mutant gene in which at least one amino acid codon at a given site of the protein-encoding gene has been mutated into an amber or ochre mutation is provided within an expression plasmid.

40. The kit according to claim 30, wherein said kit provides template DNA containing a mutant gene in a reporter construct.

41. The kit according to claim 30, wherein said reporter construct facilitates quantitative assessment of protein synthesis efficiency using said kit.

42. The kit according to claim 30, wherein said crowding reagent is polyethylene glycol (PEG).


SEQUENCE LISTING (7 sequences)

SEQ ID NO: 1 - DNA, 3202 bp, Artificial Sequence
  plasmid
aataattttgtttaactttaagaaggagatataccatggagcttttcactggcgttgttcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatctaactcgagcaaagcccgccgaaaggcgggcttttctgtgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctggcgaatcctctgaccagccagaaaacgacctttctgtggtgaaaccggatgctgcaattcagagcggcagcaagtgggggacagcagaagacctgaccgccgcagagtggatgtttgacatggtgaagactatcgcaccatcagccagaaaaccgaattttgctgggtgggctaacgatatccgcctgatgcgtgaacgtgacggacgtaaccaccgcgacatgtgtgtgctgttccgctgggcatgctgagctaacaccgtgcgtgttgacaattttacctctggcggtgataatggttgcagctagc

SEQ ID NO: 2 - DNA, 678 bp, Artificial Sequence
  reporter
atggagcttttcactggcgttgttcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatctaa

SEQ ID NO: 3 - DNA, 7036 bp, Artificial Sequence
  plasmid
tcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttggtacccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcactcggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctaacaggaggaattagatctatggataaaaaaccactaaacactctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatttctaccaacctgtaagtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttgtttgtgagctcccggtcatcaatcatccccataatccttgttagattatcaattttaaaaaactaacagttgtcagcctgtcccgctttaatatcatacgccgttatacgttgtttacgctttgaggaatcccatatggataaaaaaccactaaacactctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatttctaccaacctgtaactgcagtttcaaacgctaaattgcctgatgcgctacgcttatcaggcctacatgatctctgcaatatattgagtttgcgtgcttttgtaggccggataaggcgttcacgccgcatccggcaagaaacagcaaacaatccaaaacgccgcgttcagcggcgttttttctgcttttcttcgcgaattaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagatgcgccccgcatttatgcatggcgatatctaatacgactcactataggaaacctgatcatgtagatcgaatggactctaaatccgttcagccgggttagattcccggggtttccgccaaattcgaaaagcctgctcaacgagcaggcttttttgcatgctcgagcagctcagggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttcct

SEQ ID NO: 4 - DNA, 1365 bp, Methanosarcina mazeii
atggataaaaaaccactaaacactctgatctctgctactggtctgtggatgagtcgtaccggaaccattcataaaatcaaacaccacgaggttagccgttcgaaaatctatattgagatggcgtgtggcgatcatctggttgtgaacaatagccgctcttctcgtacagcacgtgcactgcgtcaccacaaatatcgtaaaacctgtaaacgttgccgtgtgtccgatgaggatctgaacaaattcctgacaaaagccaatgaggaccaaacaagcgtgaaagtgaaagtcgttagcgctcctacccgtactaaaaaagcaatgccgaaatccgttgctcgtgcccctaaaccactggaaaacactgaagcagcacaggcacagccgtctggaagcaaattctctccggccattcctgtttctacccaggagtccgtttctgttccagcaagtgtgagcaccagcattagcagtattagcaccggtgccaccgctagcgccctggttaaaggcaataccaatccgattacaagcatgtctgccccggttcaagcatcagctccagcactgacaaaatcccaaaccgatcgtctggaggttctgctgaatccgaaagacgaaatcagcctgaattccggcaaaccgtttcgtgaactggagagcgaactgctgtcacgtcgtaaaaaagacctgcaacaaatctatgccgaagaacgtgagaactatctggggaaactggaacgtgaaatcacccgctttttcgtggatcgtggctttctggagatcaaatccccgattctgattcctctggagtatatcgagcgtatgggcatcgacaatgataccgaactgagcaaacaaattttccgtgtggataaaaacttctgtctgcgccctatgctggcaccaaatctgtataactatctgcgcaaactggaccgtgccctgcctgatcctatcaaaatcttcgagatcggcccgtgttatcgtaaagagtccgacggtaaagaacatctggaggagtttaccatgctgaacttttgccaaatgggttcaggttgtactcgtgagaacctggaaagcatcatcaccgattttctgaaccacctgggcattgacttcaaaattgtgggcgacagctgtatggtgtatggcgacaccctggatgtcatgcacggcgacctggaactgtctagtgccgttgttggaccaattccgctggaccgtgagtggggtatcgacaaaccgtggatcggagcaggattcggtctggaacgcctgctgaaagtgaaacacgacttcaaaaacatcaaacgtgccgcccgttctgaatcgtattataacgggatttctaccaacctgtaa

SEQ ID NO: 5 - DNA, 72 bp, Methanosarcina mazeii
ggaaacctgatcatgtagatcgaatggactctaaatccgttcagccgggttagattcccggggtttccgcca

SEQ ID NO: 6 - DNA, 1152 bp, Zymomonas mobilis
atggcttcttcaactttttatattcctttcgtcaacgaaatgggcgaaggttcgcttgaaaaagcaatcaaggatcttaacggcagcggctttaaaaatgccctgatcgtttctgatgctttcatgaacaaatccggtgttgtgaagcaggttgctgacctgttgaaaacacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgctttgaaagcagcttccatgatcgctaagaatctgaagaccgcttgcgacaacggtaaggatatgccagctcgtgaagctatggcttatgcccaattcctcgctggtatggccttcaacaacgcttcgcttggttatgtccatgctatggctcaccagttgggcggttactacaacctgccgcatggtgtctgcaacgctgttctgcttccgcatgttctggcttataacgcctctgtcgttgctggtcgtctgaaagacgttggtgttgctatgggtctcgatatcgccaatctcggcgataaagaaggcgcagaagccaccattcaggctgttcgcgatctggctgcttccattggtattccagcaaatctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaa

SEQ ID NO: 7 - DNA, 1430 bp, Escherichia coli
atggcagaacgcccaacgttaccgatccctgatttgctcacgaccgatgcccgtaatcgcattcagttaactattggcgcaggccagtccacctttggcgggaaaactgcaactacctggggctataacggcaatctgctggggccggcggtgaaattacagcgcggcaaagcggtaacggttgatatctacaaccaactgacggaagagacaacgttgcactggcacgggctggaagtaccgggtgaagtcgacggcggcccgcagggaattattccgccaggtggcaagcgctcggtgacgttgaacgttgatcaacctgccgctacctgctggttccatccgcatcagcacggcaaaaccgggcgacaggtggcgatggggctggctgggctggtggtgattgaagatgacgagatcctgaaattaatgctgccaaaacagtggggtatcgatgatgttccggtgatcgttcaggataagaaatttagcgccgacgggcagattgattatcaactggatgtgatgaccgccgccgtgggctggtttggcgatacgttgctgaccaacggtgcaatctacccgcaacacgctgccccgcgtggttggctgcgcctgcgtttgctcaatggctgtaatgcccgttcgctcaatttcgccaccagcgacaatcgcccgctgtatgtgattgccagcgacggtggtctgctacctgaaccagtgaaggtgagcgaactgccggtgctgatgggcgagcgttttgaagtgctggtggaggttaacgataacaaaccctttgacctggtgacgctgccggtcagccagatggggatggcgattgcgccgtttgataagcctcatccggtaatgcggattcagccgattgctattagtgcctccggtgctttgccagacacattaagtagcctgcctgcgttaccttcgctggaagggctgacggtacgcaagctgcaactctctatggacccgatgctcgatatgatggggatgcagatgctaatggagaaatatggcgatcaggcgatggccgggatggatcacagccagatgatgggccatatggggcacggcaatatgaatcatatgaaccacggcgggaagttcgatttccaccatgccaacaaaatcaacggtcaggcgtttgatatgaacaagccgatgtttgcggcggcgaaagggcaatacgaacgttgggttatctctggcgtgggcgacatgatgctgcatccgttccatatccacggcacgcagttccgtatcttgtcagaaaatggcaaaccgccagcggctcatcgcgcgggctggaaagataccgttaaggtagaaggtaatgtcagcgaagtgctggtgaagtttaatcacgatgcaccgaaagaacatgcttatatggcgcactgccatctgctgga